(19) United States
(12) Patent Application Publication
(10) Pub. No.: US 2022/0257546 A1
(43) Pub. Date: Aug. 18, 2022

(54) KETONE BODIES ENCLOSED IN MICROBEADS

(71) Applicant: Ketoswiss AG, Basel (CH)

(72) Inventors: Elena GROSS, Basel (CH); Sinead Brigid BLEIEL, Dublin (IE)

(21) Appl. No.: 17/609,857

(22) PCT Filed: May 8, 2020
(86) PCT No.: PCT/EP2020/062929

§ 371 Date: Nov. 9, 2021

(30) Foreign Application Priority Data
May 10, 2019 (EP) 19173881.4
Oct. 22, 2019 (EP) 19204621.7

(51) Int. Cl. A61K 31/19; A61K 9/16

(52) U.S. Cl. CPC A61K 31/19; A61K 9/1694; A61K 9/1658

(57) ABSTRACT

The invention relates to a composition comprising ketone bodies contained in microbeads, in particular, for use in maintaining an increased blood concentration of ketone bodies in a subject for a prolonged period of time, preferably for the use as a medicament, food for special medical purposes/medical food or nutraceutical/food supplement. It further relates to methods to produce a composition comprising ketone bodies contained in microbeads. Also provided are non-medical uses of microbeads/ketone bodies/compositions of the invention.

The invention relates to a composition comprising ketone bodies contained in microbeads, in particular wherein said composition is suitable for maintaining an increased ketone body concentration in the blood plasma of a subject for a prolonged period of time, in particular for use in maintaining an increased blood concentration of ketone bodies in a subject for a prolonged period of time, and/or for the use as a medicament, food for special medical purposes/medical food, or a nutraceutical/food supplement. It further relates to methods to produce a composition comprising ketone bodies contained in microbeads. Also provided are non-medical uses of microbeads/ketone bodies/compositions of the invention, in particular wherein the inventive microbead or composition is comprised in a food product, nutraceutical and/or food supplement.

### BACKGROUND

Ketosis is a metabolic state in which some of the body's energy supply comes from ketone bodies in the blood, in contrast to a state of glycolysis, in which blood glucose provides energy. It is characterized by serum concentrations of ketone bodies over 0.5 mM. There are various ways to achieve ketosis which has been found to be beneficial in the treatment of a series of diseases. Ketosis is a condition which commonly occurs during starvation. During periods of carbohydrate deprivation (and protein limitation), the body utilizes energy obtained from the metabolism of fats. During fat metabolism, fats are converted to acetoacetate and 3-hydroxybutyric acid (βHB) in the liver, which are known as ketone bodies (KB), and large quantities of these substances accumulate in the blood. When blood ketone body concentrations are elevated to levels found in prolonged starvation, they provide the major source of energy for the brain (discussed in WO2004108740A2).

The ketogenic diet (KD) was developed about 100 years ago after the observation that prolonged fasting has anticonvulsive properties. With its high fat, low carbohydrate and protein content it simulates the metabolic effects of starvation. KD has been shown to be an effective alternative when treating refractory epilepsy. However, a strict KD is unlikely to provide a feasible long-term solution for many patient populations, because it can be difficult to implement in an ambulatory setting and patient adherence may be limited (discussed in WO2018115158A1).

An alternative means to induce a state of mild to medium nutritional ketosis, irrespective of dietary carbohydrate and protein intake, is the dietary supplementation with exogenous ketogenic substances, such as middle chain triglycerides (MCTs), ketogenic amino acids, βHB or AcAc supplements and more recently keto esters (βHB and/or AcAc esterified with one another). Dietary supplementation of KB themselves does not require the limitation of carbohydrate and protein, thus increasing the chance of compliance, particularly since carbohydrate diets are common in most cultures. Earlier studies in humans using MCTs suggest that those are safe, but in higher therapeutic doses not well tolerated due to strong gastrointestinal upset and many ketone esters have the problem of a very foul taste (discussed in WO2018115158A1).

To date, specific ketone esters (disclosed in WO2010021766A1) are a common exogenous source of ketone bodies because they effectively increase the blood concentration of D-β-Hydroxybutyrate (βHB), an endogenous form of a ketone body, but often have a very bad taste. Racemic βHB salts are associated with gastrointestinal problems and also have a bad taste. D-βHB salts are better tolerated, but only transiently increase the ketone body concentration in the blood upon ingestion. It is thought that supplementation with exogeneous D-βHB or a precursor is generally useful for the treatment of many disorders and has positive health effects. Administration of an exogenous source of D-βHB allows establishing increased D-βHB levels in the blood while at the same time lowering free fatty acid concentrations in the circulation, in contrast to a ketogenic diet; see Stubbs et al. 2017, Front Physiol. 2017 Oct. 30; 8:848. In addition of being an energy substrate, βHB has been shown to act as a signaling molecule regulating transcription and the epigenetic state of the cells and conferring substantial protection against oxidative stress and possibly act in an anti-inflammatory way; Shimazu et al., Science. 2013 Jan. 11; 339(6116):211-4; Simeone et al., Neuropharmacology. 2018 May 1; 133:233-241. It has been also shown in animal studies that βHB increased the brain metabolic efficiency and altered levels of neuropeptides in the brain; WO2010021766A1. Without being bound by theory, the metabolic and signaling changes induced by an exogenous βHB source may be the basis for the previously observed positive effects for the treatment of neurodegenerative disorders, in particular Alzheimer's disease; Henderson, Diet and Nutrition in Dementia and Cognitive Decline 2015, Pages 447-456; Hertz et al., J Neurochem. 2015 July; 134(1):7-20. Furthermore, it is believed that βHB may improve cognitive functions also in healthy individuals. It is also thought that βHB can effectively be used for the treatment of migraine; WO2018115158A1. Supplementation with βHB is further thought to improve athletic performance and reduce body weight; Evans et al., J Physiol. 2017 May 1; 595(9):2857-2871; WO2004108740A2; WO2010021766A1.

It is apparent that establishing nutritional ketosis with exogenous ketone bodies, preferably an exogenous source of D-βHB, may be used for different purposes. To date, the great potential of ketone bodies, in particular βHB, is not fully exploited because current compositions and/or dosage forms lead to highly variable blood concentrations over time with temporarily unnecessarily high or ineffective levels and/or lack satisfying sensory properties. This not only reduces the efficacy of ketone bodies but may also increase adverse side-effects like gastrointestinal upset (Clarke et al., Regul Toxicol Pharmacol. 2012 August; 63(3):401-8; WO2018115158A1), reduce compliance of patients and/or limit their broader use. A sustained or controlled release dosage is thus required to maintain a physiologically effective and at the same time well-tolerated ketone body blood concentration.

Accordingly, there is a need for a composition which can be used to provide ketone bodies in a sustained or controlled release dosage to a subject. In particular such a composition should be able to maintain a ketone body blood concentration at a physiologically effective level for a prolonged period of time in a subject when administered to said subject, preferably with good tolerance and sensory properties. The above technical problem is solved by the embodiments as defined in the claims.

### DESCRIPTION

Accordingly, the invention relates to a composition comprising one or more ketone bodies contained in a microbead. In particular, the inventive composition is suitable for increasing the ketone body concentration in the blood plasma to a physiologically effective extent, in particular wherein the active substance of said composition is said one or more ketone bodies.

The invention is, at least partly, based on the surprising discovery that a ketone body and/or a pharmaceutically acceptable salt thereof, can be enclosed in a microbead and the proportion of said ketone body and/or a pharmaceutically acceptable salt thereof, contained in said microbead may represent up to at least 85% or 90% of the weight of said microbead. It may be known to a person skilled in the art that, in many cases, a large amount of a ketone body must be delivered to a subject to have a therapeutically relevant or desired physiological effect in said subject. It may be also known to the skilled person that encapsulation of one or more ketone bodies in a microbead may, in many cases, dilute the amount of said ketone bodies to an extent that administration of a prohibitively large amount of said microbead to a subject would be necessary to induce the desired physiological effect in said subject.

It was thus another surprising discovery, as illustrated in the appended Examples, that administration of a ketone body contained in a microbead to a subject can increase the blood plasma concentration of said ketone body to a physiologically effective extent and/or level in said subject. It was further a surprising discovery that administration of a ketone body contained in a microbead to a subject can maintain the blood plasma concentration of said ketone body in said subject at a physiologically effective concentration for a prolonged period of time. As shown in Example 2, administration of a microbead containing beta-hydroxybutyrate (βHB) to human subjects was shown to increase βHB plasma levels to a physiologically effective plasma concentration and maintain it at said concentration for more than 1 hour and up to about 7 to 10 hours in said subjects. This is considerably longer compared to βHB not contained in a microbead, e.g. the formulation disclosed in WO2018115158A1. Further surprisingly, in a further trial, ingestion of a slightly higher dose of said microbead increased the plasma concentrations of both relevant ketone bodies, βHB and acetoacetate, in human subjects to a physiologically effective level and maintained them at said level for at least about 12 h (Example 3). Astonishingly, the maintenance of said physiologically effective, in particular therapeutically effective, βHB and acetoacetate plasma concentrations was prolonged by at least 8 hours, 9 to 10 hours or more, which was least 3-fold longer compared to the maintenance time achieved upon ingestion of non-enclosed (free) βHB. Such a long-term and stable maintenance of a physiologically effective ketone body concentration in the blood may so far only be achieved by directly infusing the ketone body into the blood stream of a subject. However, infusion with a needle usually requires the presence at a clinical site and severely restricts the freedom and life quality of the subject. Evidently, taking in about two or three daily doses of the microbead or composition of the present invention, e.g. orally, is much preferable over long-term and/or permanent infusion of a ketone body in a hospital.

It was further shown in the appended examples that the composition of the invention has good sensory properties and is well tolerated by said subjects at both doses assayed. This finding was particularly surprising since it is well known to a person skilled in the art, and as also observed in a trial described herein, that many ketone bodies have a very bad smell and/or taste and/or can cause gastrointestinal (GI) distress. In particular, the same dose of free βHB caused considerable GI distress, whereas βHB enclosed in microbeads was well tolerated (see Example 3).

Accordingly, as the person skilled in the art will appreciate, the composition/microbeads/ketone bodies of the invention lead to maintenance of a physiologically effective plasma concentration of a ketone body for a prolonged period of time in a subject by administering to said subject a microbead containing a ketone body, e.g. βHB, which is a typical and exemplary ketone body used to achieve nutritional ketosis.

Without being bound by theory, the surprising observation that a ketone body blood concentration can be maintained for up to 7 to 10 or even 12 hours at a physiologically effective level, may be, at least partly, due to a prolonged retention of the microbead containing said ketone body in the intestine of a subject upon administration of said microbead to said subject compared to administration of a non-enclosed ketone body. Another possible reason might be an improved uptake of a ketone body released from a microbead in the intestine compared to a free-floating ketone body. Without being bound by theory, a surprisingly stable ketone body blood concentration upon intake of a microbead containing said ketone body might be due to a predominant role of the release of said ketone body from said microbead in controlling the ketone body blood concentration compared to other factors such as food intake or digestion.

Again, without being bound by theory, the low level of gastrointestinal distress reported by subjects upon intake of said microbead, may be due to the release of the ketone body at many different locations in the intestine at various time-points which reduces the probability for high local ketone body concentration which might be particularly irritating. Another possible reason for a low GI distress may be the reduced mineral salt content of a composition containing not only a mineral salt of βHB, but also free βHB acid, which may be used in the context of the present invention.

In particular, as provided herein, i.e. in certain embodiments of the invention, the one or more ketone bodies contained in a microbead according to the invention is/are selected from

(a) beta-hydroxybutyric acid or beta-hydroxybutyrate (βHB),

(b) acetoacetate (AcAc),

(c) a precursor of βHB and/or AcAc,

(d) a compound comprising an acetoacetyl- and/or 3-hydroxybutyrate moiety, and

(e) a pharmaceutically acceptable salt of any one of (a) to (d).

Preferably, βHB is the D-βHB enantiomer.

The formulae of β-hydroxybutyric acid (left), D-β-hydroxybutyric acid (middle) and D-β-hydroxybutyrate (right) are depicted in the following:

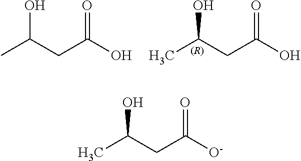

The formula of acetoacetate is depicted in the following:

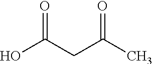

It may be known to a person skilled in the art that βHB and AcAc are the major types of ketone bodies that are produced by humans in response to fasting or a ketogenic diet. Exogenous supplementation with βHB, AcAc, a precursor of βHB and/or AcAc, and/or a compound comprising an acetoacetyl- and/or a 3-hydroxybutyrate moiety may therefore recapitulate positive health effects associated with fasting and/or a ketogenic diet.

Preferably, a compound comprising an acetoacetyl- and/or a 3-hydroxybutyrate moiety is an ester, preferably an ester of βHB and/or AcAc with one or more divalent or trivalent alcohol(s) or with a free fatty acid (FFA), i.e. a C6 to C10 free fatty acid. Suitable compounds comprising an acetoacetyl- and/or a 3-hydroxybutyrate moiety are for example, but not limited to, compounds described by any one of the formulae (Ia) to (Ve):

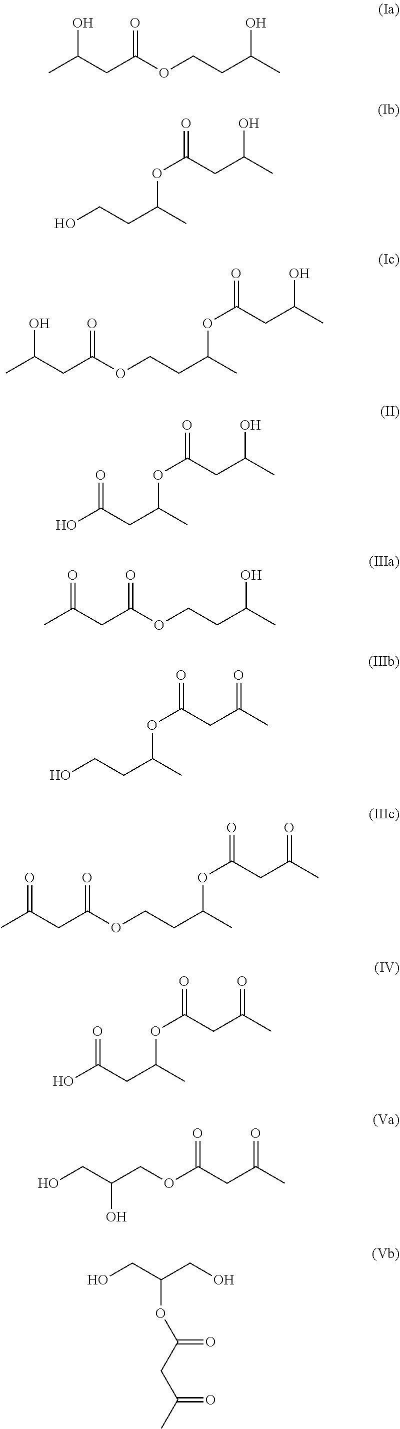

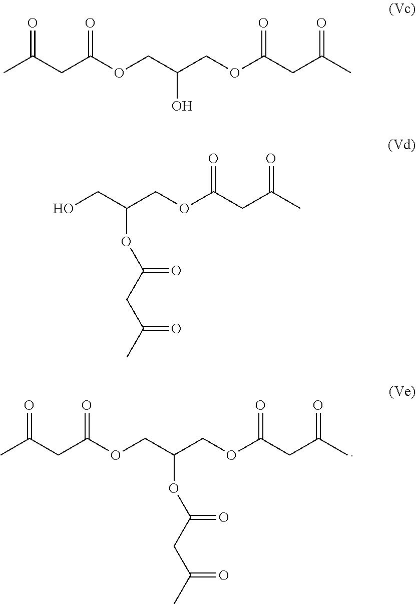

Formula (Ia) specifies 3-hydroxybutyl 3-hydroxybutanoate.

Formula (Ib) specifies (3-hydroxy-1-methyl-propyl) 3-hydroxybutanoate.

Formula (Ic) specifies 3-(3-hydroxybutanoyloxy)butyl 3-hydroxybutanoate.

Formula (II) specifies 3-(3-hydroxybutanoyloxy)butanoic acid.

Formula (IIIa) specifies 3-hydroxybutyl 3-oxobutanoate.

Formula (IIIb) specifies (3-hydroxy-1-methyl-propyl) 3-oxobutanoate.

Formula (IIIc) specifies 3-(3-oxobutanoyloxy)butyl 3-oxobutanoate.

Formula (IV) specifies 3-(3-oxobutanoyloxy)butanoic acid.

Formula (Va) specifies 2,3-dihydroxypropyl 3-oxobutanoate.

Formula (Vb) specifies [2-hydroxy-1-(hydroxymethyl)ethyl] 3-oxobutanoate.

Formula (Vc) specifies [2-hydroxy-3-(3-oxobutanoyloxy)propyl] 3-oxobutanoate.

Formula (Vd) specifies [3-hydroxy-2-(3-oxobutanoyloxy)propyl] 3-oxobutanoate.

Formula (Ve) specifies 2,3-bis(3-oxobutanoyloxy)propyl 3-oxobutanoate.

Thus, in certain embodiments, a compound comprising an acetoacetyl- and/or a 3-hydroxybutyrate moiety is an ester, wherein βHB and/or AcAc, i.e. βHB, is esterified with a free fatty acid (FFA), i.e. a C6 to C10 free fatty acid, for example, hexanoic acid, heptanoic acid, octanoic acid, nonanoic acid or decanoic acid. Such an ester may be further useful as a ketogenic agent which promotes the production of ketone bodies via the metabolism. Moreover, an ester with a FFA may be advantageous if an ester with an alcohol cannot be used.

Preferably herein, the one or more ketone bodies contained in a microbead according to the invention comprises βHB and/or a pharmaceutically acceptable salt thereof.

Thus, in a particular embodiment, the one or more ketone bodies contained in a microbead is/are βHB and/or a pharmaceutically acceptable salt thereof.

Preferably, βHB is the D-βHB enantiomer.

D-βHB or a precursor of D-βHB was shown to increase the brain metabolic efficiency and regulate the transcriptional and the epigenetic state of cells; WO2010021766A1 and Shimazu et al., Science. 2013 Jan. 11; 339(6116)211-4.

The composition of the present invention may be a microbead containing one or more ketone bodies as provided herein. In other words, the microbead according to the invention can be regarded itself as an inventive composition according to the invention. Such a microbead composition comprising a plurality of microbead particles may be in form of a powder. However, the composition of the present invention may further comprise additional components, e.g. water, i.e. when it is formulated as a suspension, and/or a further biologically active substance, or a pharmaceutically acceptable excipient or carrier. Such an additional component may be (a) contained in the same microbead particle as the one or more ketone bodies provided herein, and/or (b) in a composition according to the invention comprising (i) microbead particles containing one or more ketone bodies as provided herein and (ii) further said additional component outside of said microbead particles. In certain embodiments of the invention, i.e. in said case (b), the composition may be in form of a powder, a tablet, a capsule, or a gummy such as inter alia gummy bears.

In particular, the composition of the present invention comprises a microbead, wherein said microbead (i) contains the one or more ketone bodies provided herein, and (ii) further comprises a pharmaceutically acceptable matrix as provided herein, wherein said one or more ketone bodies is/are the active substance of said composition according to the invention and as described herein. The active substance and the matrix may be seen as two distinct compartments of the microbead which, in combination, achieve the improved physiological and/or therapeutic effects described herein in the context of the present invention. In particular, the one or more ketone bodies contained in the microbead may achieve at least a similar biological and/or therapeutic effect as has been shown previously for the supplementation with exogenous βHB or ketone esters and/or a ketogenic diet, but since said one or more ketone bodies are contained in a microbead according to the invention, said biological and/or therapeutic effect can be improved compared to state-of-the art formulations and/or dosage regimes, as illustrated in the appended Examples.

In particular, a ketone body that is used with the invention is capable of achieving a similar or better biological and/or therapeutic effect as βHB or a ketone ester/ketone body ester and/or a ketogenic diet, when administered to a subject. Surprisingly, as described herein and demonstrated in the appended Examples, the inventive composition comprising one or more ketone bodies contained in a microbead provided herein leads to an improved biological and/or therapeutic effect compared to the respective non-enclosed (“free”) ketone bodies.

In particular, the composition of the present invention, i.e. the microbeads provided herein, lead(s) to a physiologically effective increase of the ketone body concentration in the blood, when administered to a subject, as described herein in the context of the present invention. In particular, a physiologically and/or therapeutically effective concentration in the blood plasma is maintained for a prolonged period of time within the desired range as described herein in the context of the invention, i.e said physiologically effective ketone body concentration in the blood is maintained longer than what is expected for administration of said ketone body at the same dose in an, i.e. oral, immediate release dosage as described herein. Beneficial physiological, biological and/or therapeutic effects of increasing the ketone body concentration in the blood plasma and/or supplementation with an exogenous ketone body are described herein, and further beneficial effects may be known in the art.

Thus, the one or more ketone bodies provided herein comprise at most 90%, 70%, 50%, 30%, 10% or 0% poly-3-hydroxybutyrate and/or another polymerized ketone body in weight. In particular said poly-3-hydroxybutyrate and/or other ketone body consist(s) of at least 10000, 5000, 1000, 500, 200, 100, 50, 20, or 10 units of a monomeric βHB and/or other ketone body. In particular, said monomeric ketone body does not comprise more than 8, 4 or 2 βHB or AcAc units. Thus, the ketone body esters provided herein comprise preferably at least 1 or 2 βHB and/or AcAc units and/or at most 8, 4 or 3 βHB and/or AcAc units. The term “unit” refers in the context of a a polymer to a building block of said polymer, i.e. a monomer which is comprised in said polymer and/or a monomer from which said polymer is built. Thus, a ketone body, ketone ester and/or ketone body ester according to the invention is preferably not poly-3-hydroxybutyrate or poly-D-(−)-3-hydroxybutryic acid or another polyester which is used for the production of plastic such as biodegradable plastic, e.g. poly(3-hydroxybutyrate-co-3-hydroxyvalerate), or poly(3-hydroxybutyrate-co-4-hydroxybutyrate). Poly-3-hydroxybutyrate is a polyhydroxyalkanoate, and thus, a polymer belonging to the polyesters class that are of interest as bio-derived and biodegradable plastics. Poly-β-hydroxybutyrate (poly-3-hydroxybutyrate) may consist of 1000 to 30000 hydroxy fatty acid monomers. Furthermore, a ketone body, ketone ester and/or ketone body ester according to the invention is preferably also not another co-polymer of poly-3-hydroxybutyrate such as poly(β-malic acid)-b-poly(β-hydroxybutyrate).

In particular, the microbead and/or the pharmaceutically acceptable matrix of the invention does thus not consist of more than 90%, 70%, 50%, 30%, 10% or 0% poly-3-hydroxybutyrate and/or one or other polymerized ketone bodies in volume and/or weight, as described herein.

Thus, the one or more ketone bodies is/are, in particular, not exclusively contained in the pharmaceutically acceptable matrix as described herein, in particular not as an integral part or as a building block of said matrix.

In certain embodiments, the one or more ketone bodies is/are dispersed throughout a pharmaceutically acceptable matrix comprised in a microbead.

In a particular embodiment, the one or more ketone bodies is/are enriched in pockets dispersed throughout the pharmaceutically acceptable matrix.

In certain embodiments, the pharmaceutically acceptable matrix comprised in a microbead comprises yellow pea protein, soy bean protein, whey protein, gelatin, casein or EUDRAGIT® polymers, preferably yellow pea protein. In particular, said protein is denatured and/or polymerized.

In a particular embodiment, the microbead containing a ketone body comprises a polymerized and/or denatured protein matrix.

In particular, the polymerized and/or denatured protein matrix according to the invention enables the sustained release, controlled release and/or slow release of the one or more ketone bodies provided herein.

Preferably, the protein matrix essentially consists of a polymerized protein matrix, preferably a denatured protein matrix. Preferably, the denatured protein is vegetable or animal protein.

In a preferred embodiment, the matrix is a denatured pea protein matrix, preferably from yellow peas.

In one embodiment, the one or more ketone bodies contained in the microbead is/are dispersed throughout a polymerized denatured protein matrix essentially consisting of vegetable protein, preferably yellow pea protein.

In a preferred embodiment, the one or more ketone bodies contained in the microbead is/are enriched in pockets dispersed throughout a polymerized denatured yellow pea protein matrix. A microbead may have a variable number of pockets wherein a ketone body is enriched to a variable extent.

Within the present invention, a variety of different protein matrices may be used to enclose a ketone body in a microbead. Vegetable proteins are preferred, because they are well tolerated by subjects and have a high acceptance, in particular by vegetarian or vegan subjects.

In a particular embodiment, the composition of the present invention comprises one or more ketone bodies contained in a microbead as provided herein, wherein said microbead comprises a polymerized and/or denatured protein matrix as provided herein, and wherein said composition, when administered to a subject, leads to a physiologically effective increase of the ketone body concentration in the blood plasma, as described herein in the context of the present invention. Preferably, said one or more ketone bodies are dispersed throughout said pharmaceutically acceptable matrix. Preferably, said polymerized and/or denatured protein matrix comprises a vegetable protein, preferably pea protein, preferably yellow pea protein as described herein, i.e. in the context of the methods for producing a microbead according to the invention.

In one embodiment, the microbead comprises at least one core containing a ketone body and at least one shell consisting essentially of a pharmaceutically acceptable matrix, i.e. a polymer, as provided herein. It may be apparent to a person skilled in the art that the one or more cores and the one or more shells of a microbead may have different sizes, different relative locations to each other and/or be mixed or overlapping to some extent.

In certain embodiments, the average diameter of a microbead particle containing one or more ketone bodies is between 50 μm and 500 μm, preferably between 50 μm and 200 μm.

In a preferred embodiment, the average diameter of said microbead particle is between 80 μm and 150 μm.

Within the present invention, the composition comprising one or more ketone bodies contained in microbead particles within said size ranges enables delivery of the ketone body to a subject in a sustained or controlled release dosage. In particular, a composition comprising a mix of R-3-hydroxybutyric acid and a pharmaceutically acceptable salt of R-3-hydroxybutryrate contained in a matrix essentially consisting of polymerized denatured pea protein, enables the delivery of βHB to a subject in a sustained or controlled release dosage.

Preferably, the one or more ketone bodies constitute the same or a larger proportion of the microbead than the pharmaceutically acceptable matrix, e.g. at least about 50%, 70% or 90% of the weight of the microbead.

In certain embodiments, the one or more ketone bodies or a pharmaceutically acceptable salt thereof constitute(s) at least about 50%, 70%, 85% or 90% of the weight of the microbead.

In a preferred embodiment, the one or more ketone bodies or a pharmaceutically acceptable salt thereof constitute(s) at least about 80 to 90% of the weight of the microbead.

In certain embodiments, the one or more ketone bodies and/or a pharmaceutically acceptable salt thereof, constitute(s) at most 99% or 95% of the weight of the microbead.

In a particular embodiment, the one or more ketone bodies and/or a pharmaceutically acceptable salt thereof, constitute(s) at least about 50%, 70%, 85% or 90%, preferably at at least about 80 to 90% of the weight of the microbead, and at most 99% or 95% of the weight of the microbead.

Exogenous ketone bodies are often only physiologically and/or clinically effective when a large quantity of them are consumed/administered. A low ratio of ketone body weight over microbead weight may hinder the intake of the required amount of the microbead comprising one or more ketone bodies. In consequence, a physiologically effective ketone body blood concentration using a ketone body comprised in a microbead may only be achieved, at least for certain intended purposes, when said ketone body constitutes a major proportion, preferably at least 80-90% of said microbead.

In particular, the one or more ketone bodies are released from the inventive microbead provided herein in a sustained and/or a controlled way upon administration to a subject. In other words, the microbead of the invention containing the one or more ketone bodies, as such, is formulated in a sustained or controlled release dosage. Thus, said inventive microbead and/or the composition comprising said microbead allows to maintain a physiologically, biologically and/or therapeutically effective ketone body concentration in the blood plasma for a prolonged period of time as described herein. Preferably, said release is a controlled release, i.e. wherein the blood concentration of a ketone body, i.e. βHB, fluctuates around an optimal concentration within the desired range for a prolonged period of time as described herein.

In certain embodiments, any of the compositions comprising one or more ketone bodies contained in a microbead is formulated for administration to a subject in a sustained or controlled release dosage.

In a preferred embodiment, the formulation is a controlled release dosage and the blood concentration of a ketone body fluctuates around an optimal concentration within the desired range for a prolonged period of time.

Within the present invention, a sustained release dosage or a controlled release dosage allows to maintain the level of the active substance in the blood above the minimum effective concentration (MEC) for a prolonged period of time compared to an immediate release dosage. Further, a controlled release dosage allows to maintain the blood concentration of said active substance within a desired range for a prolonged period of time as described herein.

In certain embodiments, any of the compositions comprising one or more ketone bodies contained in a microbead of the invention is formulated for administration to a subject orally and/or as a suspension. Oral administration of a compound is one of the preferred dosage forms. Ketone bodies, however, often need to be taken up in such a large quantity to be physiologically effective that many oral dosage forms such as tablets, capsules, pills or lozenges, and practical non-oral dosage forms like suppositories are futile. Administration of ketone bodies as a solution or suspension may be appropriate, but at least some ketone bodies may be difficult to dissolve or suspend in aqueous solutions, may not be stable therein and/or have a foul taste. Furthermore, ketone bodies in powder form, e.g. β-hydroxbutyric acid (free acid βHB), may be hygroscopic which complicates their storage and might affect their physical and/or chemical integrity. Without being bound by theory, maintenance of a physiologically effective ketone body blood concentration upon intake of one or more ketone bodies contained in a microbead of the invention may be, at least partially, mediated by the protective effect of the matrix surrounding said ketone body in said microbead. Without being bound by theory, a ketone body enclosed in a microbead of the invention may be protected against various environmental influences which might otherwise alter its pharmacological properties. Such environmental influences are for example, but not limited to, the pH, salt concentration, reactive compounds present in a solution in which said ketone body is poured for administration and/or in the gastrointestinal tract upon intake by a subject.

In particular, the inventive composition and/or microbead provided herein may enable the delivery of the one or more ketone bodies contained therein in their intact, i.e. chemically and/or physically unaltered, form to the small intestine, i.e. the ileum, of a mammalian subject, i.e. upon oral intake.

In a particular embodiment of the invention, a composition comprising one or more ketone bodies contained in a microbead is formulated for intact delivery to the small intestine of a mammalian subject upon oral intake. Preferably, said microbead is capable of remaining intact during gastric transit and a ketone body comprised therein is released in the small intestine, specifically in the ileum. For example, in one embodiment, a ketone body is delivered without damage to the small intestine because it is contained in a microbead.

In certain embodiments of the invention, any of the compositions comprising one or more ketone bodies contained in a microbead is formulated as powder or tablet, preferably for dissolution in the mouth and/or an aqueous solution.

In certain embodiments of the invention, any of the compositions comprising one or more ketone bodies contained in a microbead may be suspended in an aqueous solution. Preferably, said compositions are at least as long stable in an aqueous solution that a subject can comfortably take in the suspension.

In certain embodiments of the invention, the composition comprising one or more ketone bodies contained in a microbead is formulated for administration in doses between 0.01 g/kg body weight and 1 g/kg body weight.

In a particular embodiment of the invention, the composition comprising βHB contained in a microbead is formulated for administration in doses between 0.1 g/kg body weight and 0.4 g/kg body weight.

In a particular embodiment of the invention, a dose is formulated for administration once a day, twice a day, three times a day, four times a day or several times a day. The administered dose and/or the frequency of administration depends on the intended purpose and/or the subject to which the composition is administered.

In certain embodiments of the invention, any of the aforementioned compositions is an “off-the-shelf” product. In particular, said “off-the-shelf” product allows adjusting the dose by ordinary means, for example by measuring the amount of the composition with scales, a measuring cup or a spoon.

In certain embodiments of the invention, any of the aforementioned compositions, when administered to a subject, leads to a physiologically effective increase of the ketone body concentration in the blood plasma of said subject.

It was shown in an example disclosed herein, which however does not limit the scope of the invention, that administration of 15 g of a composition comprising D-βHB contained in a microbead to a human subject led to a significant and physiologically effective increase of the βHB concentration in the blood of said subject. The average measured peak βHB blood concentration was about 1.1 mM which complies with previously suggested optimal or preferred βHB concentration ranges; WO2010021766A1 and Evans et al., J Physiol. 2017 May 1; 595(9):2857-2871. As the skilled person will appreciate, the optimal dose may depend on the particular ketone body and/or the intended use.

In particular, the ketone body concentration in the blood plasma as provided herein and in the context of the present invention may be the concentration of βHB and/or AcAc in the blood plasma. Preferably herein, the ketone body concentration in the blood plasma is the concentration of βHB, i.e. D-βHB, in the blood plasma.

Even if only other ketone bodies than βHB or AcAc, e.g. a precursor of βHB and/or AcAc and/or a compound comprising an acetoacetyl- and/or 3-hydroxybutyrate moiety, e.g. an ester as described herein, are contained in the microbead and/or composition of the invention, the ketone body concentration in the blood plasma is preferably the concentration of βHB and/or AcAc in the blood plasma, preferably the concentration of βHB, i.e. D-βHB.

A therapeutically relevant ketone body blood concentration may range from less than 0.2 mM to more than 4 mM (see van Hove et al., Lancet 2003, 361, 1433-1435 and Gilbert et al., J. Child Neurol. 2000, 15, 787-790).

In certain embodiments, a ketone body blood concentration between about 0.3 mM and about 5 mM is achieved upon administration of the compositions provided herein. Preferably, said ketone body is βHB, more specifically, D-βHB.

In certain embodiments, a ketone body blood concentration between about 0.5 mM and about 1.5 mM is achieved upon administration of the compositions provided herein. Preferably, said ketone body is acetoacetate.

As βHB is metabolized into acetoacetate (AcAc) in the body, increasing and maintaining increased blood concentrations of both ketone bodies, βHB and AcAc, is therapeutically relevant.

In a particular embodiment, a ketone body blood concentration between about 0.8 mM and about 2 mM is achieved upon administration of the compositions provided herein. Preferably, said ketone body is βHB, more specifically, D-βHB.

In the context of the present invention, a physiologically effective increase of the ketone body concentration in the blood plasma upon administration of the composition and/or microbead of the invention may be an at least 1.8 fold, preferably at least 2.4 fold, preferably at least 3-fold higher ketone body concentration than the baseline concentration (i.e. before said administration), and, in particular, said increase results in a physiologically effective concentration, i.e. a concentration between 0.3 mM and 5 mM, in particular between 0.5 mM and 3 mM, e.g. between 1 mM and 3 mM, or preferably between 0.5 mM and 2 mM, in particular between 0.5 mM and 1.5 mM or between 0.8 mM and 2 mM. Preferably, said ketone body in the blood plasma is βHB, more specifically, D-βHB.

In certain embodiments, the compositions provided herein, when administered to a subject, maintains the ketone body concentration in the blood plasma for a prolonged period of time at a physiologically effective concentration, in particular between 0.3 mM and 5 mM, preferably between 0.5 mM and 1.5 mM or between 0.8 mM and 2 mM, in particular for 2, 4, 6, 8, 10, 12 or 14 hours as described herein. Preferably, said physiologically effective ketone body concentration in the blood plasma is at least 2-fold, preferably at least 3-fold longer maintained, for example by 8, 9, or 10 hours, in comparison to the corresponding non-enclosed (“free”), ketone body/ies, as described herein.

In a particular embodiment, the compositions, when administered to a subject, maintains the ketone body concentration in the blood plasma for about 2, 4, 6, 8, 10, 12 or 14 hours at a physiologically effective concentration, as described herein.

The time for which a physiologically effective ketone body plasma concentration is maintained upon ingestion of a composition provided herein comprising one or more ketone bodies contained in a microbead can be controlled by the amount of the pharmaceutically acceptable matrix material comprised in a microbead particle. Without being bound by theory, if a microbead particle comprises proportionally more matrix material and/or has a larger size, the ketone body plasma concentration will be maintained for a longer time.

In a preferred embodiment, said concentration is within the desired range which is between the minimum effective concentration (MEC) and the maximum tolerated concentration (MTC). Preferably, the ketone body concentration in the blood may fluctuate around the desired concentration, but it does not go below the MEC or above the MTC for a time period during said 2, 4, 6, 8, 10, 12 or 14 hours which is considered by a skilled person to be relevant, for example, it is not outside the MEC or MTC for more than 4 hours, 2 hours or, preferably, 1 hour. Preferably, said ketone body is βHB, more specifically, D-βHB. In particular, the MEC of βHB, i.e. D-βHB, is 0.3 mM, preferably 0.5 mM, preferably 0.8 mM, preferably 1 mM and the MTC of βHB is 8 mM, preferably 5 mM, preferably 3 mM, preferably 2 mM.

In a preferred embodiment, the compositions of the invention, when administered to a subject, maintain the ketone body concentration in the blood plasma for about 7 to 8 hours at a physiologically effective concentration, preferably within said desired range. Preferably, the time is about 8 hours. Preferably, said ketone body is βHB, more specifically, D-βHB. Thus, such a composition is preferably taken in three times a day to maintain a physiologically effective concentration, i.e. constant therapeutic ketosis.

In a particular embodiment, the compositions provided herein, when administered to a subject, maintain the ketone body concentration in the blood plasma for about 11 to 14 hours at a physiologically effective concentration, preferably within said desired range. Preferably, the time is about 12 hours. Preferably, said ketone body is βHB, more specifically, D-βHB. Thus, such a composition is preferably taken in two times a day to maintain a physiologically effective concentration, i.e. constant therapeutic ketosis. In a particular embodiment, the compositions provided herein, when administered to a subject, maintain the ketone body concentration in the blood plasma for about 11 to 14 hours at a physiologically effective concentration, preferably within said desired range. Preferably, the time is about 12 hours. Preferably, said ketone body is βHB and/or acetoacetate, preferably βHB, and preferably wherein βHB is more specifically D-βHB.

In one embodiment, the compositions provided herein, when administered to a subject, maintain the ketone body concentration in the blood plasma for about 10 hours at an increased concentration compared to the concentration before ingestion of said composition. Preferably, said ketone body is βHB, more specifically, D-βHB.

In a particular embodiment, the compositions provided herein, when administered to a subject, lead to a ketone body concentration in the blood plasma, between about 0.8 mM and about 2 mM from about 30 min to about 8 hours after ingestion of said composition, preferably wherein said ketone body is βHB, more specifically, D-βHB.

In a particular embodiment, the compositions provided herein, when administered to a subject, lead to a ketone body concentration in the blood plasma, between about 0.8 mM and about 2 mM from about 30 min to about 12 hours after ingestion of said composition, preferably wherein said ketone body is βHB, more specifically, D-βHB.

In a particular embodiment, the compositions provided herein, when administered to a subject, lead to a ketone body concentration in the blood plasma, between about 0.5 mM and about 1.5 mM from about 30 min to about 12 hours after ingestion of said composition, preferably wherein said ketone body is acetoacetate.

In a particular embodiment, the compositions provided herein, when administered to a subject, lead to a ketone body concentration in the blood plasma, between about 0.8 mM and about 2 mM from about 30 min to any time between about 11 and 14 hours, preferably about 12 hours, after ingestion of said composition, preferably wherein said ketone body is βHB, more specifically, D-βHB.

In a particular embodiment, the compositions provided herein, when administered to a subject increase the time for which an increased ketone body concentration in the blood plasma is maintained at least 2-fold, preferably at least 3-fold. Preferably, said ketone body is βHB and/or acetoacetate, preferably βHB, and preferably wherein βHB is more specifically D-βHB.

In a particular embodiment, the compositions provided herein, when administered to a subject prolong the time for which an increased ketone body concentration in the blood plasma is maintained by at least 3 hours, preferably at least 6 hours, preferably at least 8 hours. Preferably, said ketone body is βHB and/or acetoacetate, preferably βHB, and preferably wherein βHB is more specifically D-βHB.

Without being bound by theory, an increased ketone body blood plasma concentration is maintained for a prolonged period of time because of the specific properties of any of the aforementioned compositions. For example, a non-fully synchronized degradation of microbead particles in the small intestine, in particular in the ileum, and/or a gradual release of the active compound from a microbead particle may lead to a release of a ketone body for a prolonged period of time in this organ.

A microbead as provided herein may be produced by a method comprising a step of enclosing a ketone body in a pharmaceutically acceptable matrix.

In certain embodiments, the pharmaceutically acceptable matrix comprised in a microbead is generated by a polymerization process.

In a particular embodiment, a polymer essentially consisting of a denatured animal or vegetable protein is generated during said polymerization process. Preferably, said denatured protein is pea protein, preferably, yellow pea protein.

In certain embodiments, the production of a microbead comprises the steps of (a) preparing a protein solution, solubilising the protein and denaturing the protein, (b) generating a dispersion by combining the denatured pea protein solution with a ketone body and/or a pharmaceutically acceptable salt thereof, extruding the dispersion through an orifice forming microdroplets that free-fall into a polymerisation bath and curing the formed microparticulates, and (c) drying said microparticulates.

“Extrusion” typically means passing the solution through a small orifice whereby the solution is broken up into micron-sized droplets. Preferably, the solution is extruded through an orifice. Various methods will be apparent to the skilled person for generating droplets, for example prilling and spraying (ie spray drying).

A preferred method of producing the microbeads is a vibrating nozzle technique, in which the suspension is sprayed (extruded) through a nozzle and laminar break-up of the sprayed jet is induced by applying a sinusoidal frequency with defined amplitude to the spray from the nozzle. Typically, the spray nozzle has an aperture of between 50 and 600 microns, preferably between 50 and 200 microns, suitably 50-200 microns, typically 50-150 microns, and ideally about 80-150 microns. Suitably, the amplitude is from 4.7 kV to 7 kV. Typically, the falling distance (from the nozzle to the acidification bath) is less than 50 cm, preferably less than 40 cm, suitably between 20 and 40 cm, preferably between 25 and 35 cm, and ideally about 30 cm. The flow rate of suspension (passing through the nozzle) is typically from 3 to 10 L/min; an ideal flow rate is dependent upon the nozzle size utilized within the process.

In some embodiments the nozzle assembly comprises an outer nozzle disposed concentrically around an inner nozzle, in which the denatured pea protein solution is extruded through the outer nozzle and an active agent solution comprising active agent is extruded through the inner nozzle, and wherein the microparticles are microcapsules having a denatured and/or polymerized protein shell and a core comprising a ketone body.

In a particular embodiment, a ketone body is added to a denatured protein solution prior to the microdroplet forming step, wherein the nozzle assembly typically comprises a single nozzle, and wherein the microparticles are microbeads having a continuous denatured protein matrix with a ketone body distributed throughout the denatured protein matrix. Preferably, a ketone body is enriched in pockets which are distributed throughout the denatured protein matrix.

In a preferred embodiment, the production of a microbead containing at least one ketone body comprises the following steps:
Step 1:
   (a) Preparing pea protein solution in aqueous 0.1 M NaOH to a concentration of 10% to 15% w/v with a pH between 8 and 11.
   (b) Solubilising the protein by storing the solution, i.e. for 45 min, at room
   (c) Adjusting to pH 7.5-10 using e.g. HCl or NaOH/KOH as required
   (d) Heat-treating the solution to denature the protein, i.e. at a min. temperature of at least 85° C. and i.e. maintaining that temperature for a duration of min. 25 min.
Step 2:
   (a) Generating a dispersion by combining the denatured pea protein solution from step 1 with a ketone body. A particularly suitable ketone body is βHB and/or a pharmaceutically acceptable salt thereof. Preferably, the ketone body is a mix of 50% R-3-hydroxybutyric acid and 50% of a mineral salt of R-3-hydroxybutryrate, wherein the mineral salt is a blend of sodium, calcium, potassium, magnesium mineral salts at a mixing ratio of 1:2:1:2, 1:1:1:2 or 1:1:1:1.
   (b) Extruding the dispersion from (a) through an orifice forming microdroplets that free-fall into polymerization bath. Preferably, the polymerization bath is a 0.1-0.5 M citrate polymerization bath.
   (c) Curing the microparticulates formed in (b) in a polymerization buffer at low agitation speed, i.e. for 2 hours, at room temperature
Step 3:
   Drying of the microparticulates from step 2 by using a hot air circulation in a spray drier, i.e. with inlet temperatures of 183° C./outlet 92° C.

Preferably, the pea protein solution used for the production of one or more ketone bodies contained in a microbead comprises yellow pea protein. Preferably, the stock pea protein solution has a very high content of soluble pea protein, for example greater than 90%, and a sufficiently low viscosity to enable it to be extruded or sprayed through a nozzle.

For further details and embodiments of the production process and the structure of the microbeads, reference is made to WO2016096929A1. The person skilled in the art is able to perform modifications to the protocols for producing a microbead according to the invention provided herein and illustrated in the appended Examples, based on WO2016096929A1 and common general knowledge, and evaluate the slow release properties and/or the physiological effects of the microbeads, i.e. the prolonged maintenance of a physiologically effective ketone body concentration in the blood as described herein. In particular, minor modifications, for example, slightly adjusting the concentration of the protein solution before solubilization and/or the ratio of the denatured protein solution and ketone body may be readily performed.

For the enclosure of a ketone body in form of a hygroscopic powder and/or small crystals, e.g. β-hydroxybutyric acid (free acid βHB), the methods for producing the inventive microbeads provided herein may be modified to improve the stability of said microbeads. In particular, it should be avoided that a hygroscopic ketone body powder attracts water before and/or while it is dispersed in the matrix solution, i.e. the denatured protein solution described herein. For example, a food grade desiccant, i.e. silica (silicon dioxide) may be added to β-hydroxybutyric acid, i.e at 4% (w/w) or less, e.g. 3.2 to 3.4% (w/w), i.e. when the β-hydroxybutyric acid package is opened. The resulting ketone body/silica mix may then be used for production of the microbeads of the invention.

Thus, in some embodiments, the composition and/or microbead of the present inventions comprises a food grade desiccant, in particular silicon dioxide, preferably at 4% or less (w/w).

Alternatively, or in addition, the microbead of the invention may be produced in a moisture-controlled environment, i.e. the package of a hygroscopic ketone body powder may be opened in such an environment and all production steps until the ketone body is protected within the microbead, i.e. until polymerization and/or drying of the microbead, may be carried out in said moisture-controlled environment. In particular, the relative humidity in a moisture-controlled environment is at most 50%, 30%, 10% or 5%, in particular at most 30%.

Of note, in certain embodiments, silica is not added to the ketone body, i.e. when the ketone body powder is not strongly hygroscopic, e.g. a βHB (β-hydroxybutyrate) salt as provided herein, e.g., wherein the salt is a blend of sodium, calcium and potassium, magnesium as provided herein. Moreover, silica is not necessarily added when the ketone body is a mix of β-hydroxybutyric acid and a pharmaceutically acceptable salt of β-hydroxybutryrate, e.g. at a ratio of about 1:1.

In certain embodiments, the one or more ketone bodies for production of the microbeads of the present invention may be in powder form and/or in form of small crystals.

In some embodiments, the one or more ketone bodies for production of the microbeads of the present invention may be in a liquid form or dissolved in a liquid, i.e. be in solution. In particular, when the ketone body is in solution, the microbead containing said one or more ketone bodies may be produced by spray drying methods known in the art, e.g. inter alia as disclosed in WO2016096929A1. In particular, the ketone body in solution may be βHB that is dissolved in a liquid for example, the medium of a microorganism's, i.e. bacterial culture, wherein the microorganisms/bacteria, produce βHB and excrete it into the culture medium.

In general, the microbead integrity, moisture content, surface morphology and/or microbiological stability, as well as the purity of the compounds, i.e. of the enclosed one or more ketone bodies (e.g. D-βHB) may be verified by methods known in the art, e.g. as described in the appended Examples. Moreover, the functionality of the microbeads, i.e. the sustained or controlled release of the ketone body may be verified by in vitro assays, and/or by clinical trials, i.e. as described in the appended Examples.

In certain embodiments, the compositions of the present invention may be used as a medicament.

In certain embodiments, the compositions of the invention may be used as a medical food and/or food for special medical purposes.

The compositions/microbeads of the invention are provided for use in the treatment of a disorder or disease. The compositions/microbeads of the invention are improved over those provided in the prior art due to their ability for sustained or prolonged release dosage which allows maintaining an effective ketone body blood concentration in said subject for a prolonged period of time. It will be understood by a person skilled in the art, that prolonging the time of a physiologically effective ketone body blood concentration reduces the frequency in which said subject needs to take in ketone bodies to have a desired therapeutic effect. In particular, maintaining a physiologically effective ketone body blood concentration for about 6 to 12 hours upon intake of one dose of the compositions/microbeads provided herein allows maintaining a permanently increased physiologically effective ketone body blood concentration with about two to four daily doses and thus, for example, without the requirement to wake up during night. The present invention therefore greatly improves the compliance of patients to take in ketone bodies as often as necessary to achieve the desired therapeutic effect. For certain disorders or diseases, a permanently increased physiologically effective ketone body blood concentration may even be required for the optimal treatment of said disorders or diseases.

In certain embodiments, the compositions of the invention are provided for use in the treatment of neurological disorders or diseases and/or neurodegenerative disorders, as described herein in the context of a ketogenic diet and/or ketone bodies.

There are several lines of evidence that ketone bodies may be used to treat or prevent neurological disorders. It has been observed that a ketogenic diet and associated increased βHB blood levels protected children from seizures; Gilbert et al., J. Child Neurol. 2000, 15, 787-790; Zhang et al., Curr Neuropharmacol, vol. 16, no. 1, pp. 66-70, 2018. Furthermore, it has been demonstrated that a ketogenic diet may be beneficial for treating epilepsy in patients with childhood epilepsy that is characterized by an imbalance of T helper type 17 cells/regulatory T cells (Th17/Treg cells) (Ni (2016), Seizure, 38:17-22). A ketogenic diet is also thought to ameliorate symptoms in patients suffering from migraine; see for example, C. Di Lorenzo et al., European journal of neurology: the official journal of the European Federation of Neurological Societies, August 2014. Furthermore, it was shown that administration of βHB to trial participants suffering from migraine effectively decreased the frequency of migraine days of said participants; WO2018115158A1. It is therefore plausible that a composition comprising one or more ketone bodies contained in a microbead may be used for the treatment of epilepsy and/or migraine and/or symptoms associated with migraine. Previous studies indicate that a ketogenic diet may also have beneficial effects for patients suffering from autism (Castro et al., Research in Autism Spectrum Disorders, vol. 20, pp. 31-38, December 2015) and may be useful for the treatment of depression, affective disorders or anxiety (Murphy et al., Biological Psychiatry, vol. 56, no. 12, pp. 981-983, December 2004). It is further believed, that βHB may alleviate depression and anxiety by controlling the expression of brain derived neurotrophic factor (BDNF); Sleiman et al., Elife. 2016 Jun. 2; 5. pii: e15092. It is therefore plausible that a composition comprising one or more ketone bodies contained in a microbead may be used for the treatment of further neurological diseases such as autism, depression, affective disorders and anxiety. Since it is believed that ketone bodies may be beneficial for a patient suffering from traumatic brain injury (White H, Venkatesh B. Clinical review: ketones and brain injury. Crit Care. 2011 Apr. 6; 15(2):219), it is plausible that a composition comprising one or more ketone bodies contained in a microbead may be used for the treatment of traumatic brain injury.

There is also mounting evidence that ketone bodies may be used for the treatment of neurodegenerative disorders. In particular, ketone bodies are thought to be useful for the treatment of Alzheimer's disease; see for example: Henderson, Diet and Nutrition in Dementia and Cognitive Decline 2015, Pages 447-456; Hertz et al., J Neurochem. 2015 July; 134(1):7-20; Mamelak M. Energy and the Alzheimer brain. Neurosci Biobehav Rev. 2017 April; 75:297-313; Cunnane et al., Ann. N. Y. Acad. Sci., vol. 1367, no. 1, pp. 12-20, 2016. Previous studies indicate that a ketogenic diet may also have beneficial effects for patients suffering from Parkinson's disease (Phillips et al., Mov Disord. 2018 August; 33(8):1306-131) or modulate mechanisms associated with multiple sclerosis (Kim et al., PLoS ONE, vol. 7, no. 5, p. e35476, 2012; Bock et al., EBioMedicine, vol. 36, pp. 293-303, October 2018; Swidsinski et al., Front Microbiol, vol. 8, p. 1141, 2017.) It is therefore plausible that a composition comprising one or more ketone bodies contained in a microbead may be used for the treatment of further neurodegenerative diseases such as Parkinson's disease and multiple sclerosis.

Without being bound by theory, there may be several, not mutually exclusive, mechanisms, by which ketone bodies, in particular βHB, exert their positive effects for the treatment of neurological and/or neurodegenerative disorders. It is known that ketone bodies are the only alternative energy source for the brain to glucose. Thus, when neurons in the brain lack a sufficient supply with glucose, ketone bodies may rescue this lack of energy. An insufficient supply with glucose, which might occur, for example, as a result of a pathological alteration of the glucose metabolism, may especially be harmful for the poorly myelinated long axon hippocampal and cortical neurons which have a high energy demand and which are associated with Alzheimer's disease. It was also disclosed in WO2010021766A1, that βHB increased the brain metabolic efficiency in rats.

In addition to its role as energy supplier, βHB is thought to modulate gene regulatory processes in cells, in particular in the brain. βHB has been shown to act as a regulator of the epigenetic and transcriptional state of cells; Shimazu et al., Science. 2013 Jan. 11; 339(6116):211-4. Without being bound by theory, βHB can via this mode of action, modulate the level of important signaling molecules in the brain like the neuropeptide Brain Derived Neurotropic Factor (BDNF) which is associated for example with preventing apoptosis and promoting neuronal growth, enhancing mental abilities and acting against anxiety and depression, and the neuropeptide Cocaine-and-Amphetamine Responsive Transcript (CART) which is thought to promote alertness. The regulation of BDNF by βHB occurs likely via its activity as an inhibitor of histone deacetylases (HDAC) which repress the production of BDNF; Sleiman et al., Elife. 2016 Jun. 2; 5. By regulation of FOXO3A and MT2, the HDAC inhibitor βHB is also thought to confer substantial protection against oxidative stress; Shimazu et al., Science. 2013 Jan. 11; 339(6116):211-4.

Another aspect of supplementation with exogenous ketone bodies is the reduction of the level of free fatty acids circulating in the plasma which may further convey neuroprotective effects; WO2004105742A1.

It is further believed that a ketogenic diet or ketone bodies may be useful for the treatment of cancer, for example as adjuvant; Allen et al., Redox Biology, vol. 2, pp. 963-970, January 2014; Seyfried et al., Epilepsia, vol. 49, no. s8, pp. 114-116, 2008; Zhou et al., Nutrition & Metabolism, vol. 4, no. 1, p. 5, February 2007. Without being bound by theory, a shift from glycolysis to ketosis may allow to exploit the metabolic differences between cancer cells and normal cells. For example, ketosis may selectively increase metabolic oxidative stress in cancer cells and/or lead to starvation of cancer cells, in particular brain tumor cells, while maintaining a good energy supply to normal cells, in particular brain cells. It is therefore plausible that a composition comprising one or more ketone bodies contained in a microbead may be used for the treatment of cancer, in particular by exploiting the metabolic differences between cancer cells and normal cells.

Thus, the inventive compositions and/or microbeads provided herein may be further used for the treatment of cancer, as described herein in the context of a ketogenic diet and/or ketone bodies.

Previous studies suggest that a ketogenic diet or ketone bodies may be used for the treatment of metabolic disorders, in particular, genetic metabolic disorders such as Glycogen Storage Disease (Valayannopoulos et al., Pediatr Res. 2011 December; 70(6):638-41), Acyl-CoA Dehydrogenase Deficiency (Gautschi et al., Pediatr Res. 2015 January; 77(1-1):91-8; Van Hove et al., Lancet. 2003 Apr. 26; 361(9367):1433-5) and GLUT1 Deficiency Syndrome (Klepper, Epilepsy Res. 2012 July; 100(3):272-7). Without being bound by theory, supply with ketone bodies may rescue or ameliorate some of the pathological consequences of a metabolic disorder, in particular if caused by a genetic defect. It is therefore plausible that a composition comprising one or more ketone bodies contained in a microbead may be used for the treatment of metabolic disorders, for example, but not limited to, Glycogen Storage Disease, Acyl-CoA Dehydrogenase Deficiency and GLUT1 Deficiency Syndrome.

Furthermore, the inventive compositions and/or microbeads provided herein may be used for the treatment of a metabolic disorder, as described herein in the context of a ketogenic diet and/or ketone bodies.

It has been further shown that supplementation with βHB may be useful for treating patients with heart failure and reduced ejection fraction (Nielsen (2019), Circulation, 139:2129-2141). Thus, the inventive compositions and/or microbeads provided herein may be used for the treatment of a cardiovascular disease, in particular heart failure and reduced ejection fraction.

Is has been also shown that a ketogenic diet or supplementation with βHB may be useful for treating polycystic kidney disease (Torres (2019), Cell Metab., 30(6):1007-1023). Thus, the inventive compositions and/or microbeads provided herein may be used for the treatment of a kidney disease, in particular polycystic kidney disease.

Based on the herein described properties of ketone bodies, the compositions provided herein may be used for the treatment of a variety of disorders or diseases which are associated with any of the mechanism described above, for example high levels of free fatty acids, oxidative stress, a pathologically altered metabolic state, and/or transcriptional and/or epigenetic dysregulation.

To achieve a better understanding of the mode of action of ketone bodies and/or develop improved therapies for patients, it is important to control the ketone body concentration in the blood.

Moreover, the inventive compositions and/or microbeads provided herein may be used in a food product and/or as food supplements, in particular for consumption by a subject which is not suffering from any of the disorders or diseases described herein in the context of the medical uses of the inventive compositions/microbeads. Still, a rather healthy subject may seek to improve its cognitive performance, decrease food craving, decrease body weight, decrease the fat to body weight ratio, maintain or improve muscle power, and/or improve athletic performance and/or endurance by taking in the composition and/or microbeads of the invention.

In certain embodiments, the compositions comprising one or more ketone bodies contained in a microbead are comprised in a food product. The food product may further comprise any edible and/or drinkable material.

In a particular embodiment, the food product is a drink or a powder for the preparation of a drink.

In certain embodiments, any of the compositions comprising one or more ketone bodies contained in a microbead is comprised in a food supplement.

In a particular embodiment, the food supplement is a drink or a tablet or powder for the preparation of a drink and/or dissolution in the mouth.

In certain embodiments, any of said food products or food supplements may increase cognitive performance, decrease food craving, decrease body weight, decrease the fat to body weight ratio, maintain or improve muscle power, and/or improve athletic performance and/or endurance of a subject.

It may be known to a person skilled in the art that intake of a ketone body by a human subject has been shown and/or proposed to promote achieving any one or more of those features in that subject. Without being bound by theory, any health benefit may be due to any of the above discussed mechanisms. It is plausible that ketone bodies may modulate the brain function by supplying energy, alter the transcriptomic and/or epigenetic state of brain cells and/or alter cellular signals in the brain. An increase in cognitive performance and/or alteration of the eating behavior which may lead to a decreased body weight and/or a lower proportion of fat in the body, may result from the intake of a composition comprising one or more ketone bodies contained in a microbead, which occurs preferably regularly. Again, without being bound by theory, an improvement of the athletic performance and/or endurance of a subject consuming said composition, may be also caused by any of the aforementioned mechanisms, for example by utilization of ketone bodies as an energy source; Evans et al., J Physiol. 2017 May 1; 595(9):2857-2871.

Thus, the present invention further relates to:
1. A composition comprising one or more ketone bodies contained in a microbead.
2. The composition of embodiment 1, wherein the one or more ketone bodies is/are selected from
   (a) beta-hydroxybutyric acid or beta-hydroxybutyrate (βHB),
   (b) acetoacetate (AcAc),
   (c) a precursor of βHB and/or AcAc,
   (d) a compound comprising an acetoacetyl- and/or 3-hydroxybutyrate moiety, and
   (e) a pharmaceutically acceptable salt of any one of (a) to (d).
3. The composition of embodiment 2, wherein the precursor of βHB and/or AcAc and/or the compound comprising an acetoacetyl- and/or 3-hydroxybutyrate moiety is an ester.
4. The composition of any one of embodiments 2 to 3, wherein the precursor of βHB or AcAc and/or the compound comprising an acetoacetyl- and/or 3-hydroxybutyrate moiety is an ester of βHB and/or AcAc with one or more divalent or trivalent alcohol(s).
5. The composition of any one of embodiments 2 to 4, wherein the precursor of βHB and/or AcAc is 1,3-butanediol (CAS No. 107 88 0) or triacetin (CAS No. 102-76-1).
6. The composition of any one of embodiments 2 to 4, wherein the compound comprising an acetoacetyl- and/or 3-hydroxybutyrate moiety is described by any one of the formulae (Ia) to (Ve).

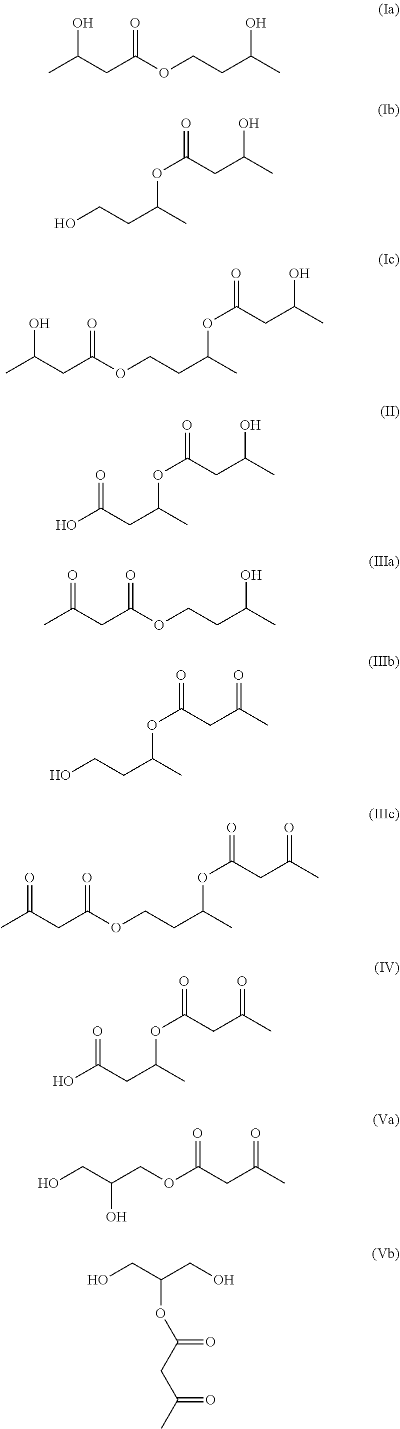

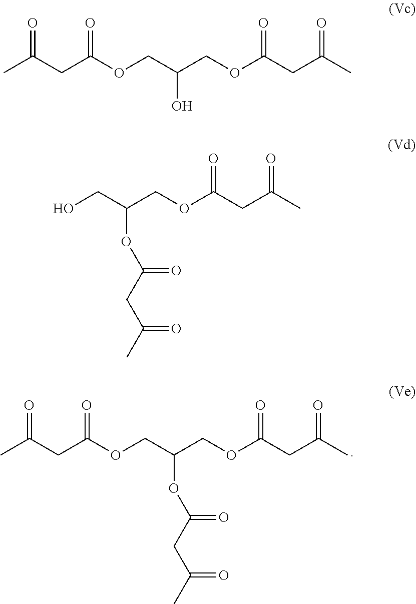

7. The composition of any one of embodiments 1 to 6, wherein the one or more ketone bodies is/are βHB and/or a pharmaceutically acceptable salt thereof.
8. The composition of any one of embodiments 2 to 7, wherein βHB is D-βHB.
9. The composition of any one of embodiments 1 to 8, wherein the one or more ketone bodies is/are AcAc, and/or a pharmaceutically acceptable salt thereof.
10. The composition of any one of embodiments 2 to 9, wherein the pharmaceutically acceptable salt is selected from a potassium salt, a sodium salt, a calcium salt, a magnesium salt, an arginine salt, a lysine salt, a leucine salt, a histidine salt, an ornithine salt, a creatine salt, an agmatine salt, a citrulline salt, a methyl glucamine salt and a carnitine salt, or a combination of said salts, in particular a combination of a calcium, a sodium, a potassium, a magnesium salt, in particular a combination of a calcium, a sodium, a potassium, a magnesium and a lysine salt.
11. The composition of any one of embodiments 1 to 10, wherein the one or more ketone bodies is/are a mix of 50% R-3-hydroxybutyric acid and 50% of a pharmaceutically acceptable salt of R-3-hydroxybutryrate.
12. The composition of embodiment 11, wherein the pharmaceutically acceptable salt is a blend of sodium, calcium, potassium, magnesium mineral salts at a mixing ratio of 1:1:1:2 or 1:1:1:1.
13. The composition of any one of embodiments 1 to 12, wherein the microbead comprises a pharmaceutically acceptable matrix.
14. The composition of embodiment 13, wherein the pharmaceutically acceptable matrix comprises polymerized protein.
15. The composition of embodiment 14, wherein the protein comprises denatured animal or vegetable protein.
16. The composition of embodiment 15, wherein the denatured protein comprises yellow pea protein.
17. The composition of any one of embodiments 1 to 16, wherein a ketone body is dispersed throughout a pharmaceutically acceptable matrix comprised in a microbead.
18. The composition of any one of embodiments 1 to 16, wherein a ketone body is locally enriched in pockets dispersed throughout a pharmaceutically acceptable matrix comprised in a microbead.
19. The composition of any one of embodiments 1 to 18 wherein at least one ketone body or a pharmaceutically acceptable salt thereof, constitutes at least about 50%, 70% or 90% of the weight of the microbead.
20. The composition of any one of embodiments 1 to 19 which is formulated as a suspension.
21. The composition of embodiment 20, wherein the suspension comprises an aqueous solution.
22. The composition of any one of embodiments 1 to 21, which is formulated for administration to a subject in a sustained release dosage or a controlled release dosage.
23. The composition of any one of embodiments 1 to 22, which is formulated for administration to a subject in a controlled release dosage.
24. The composition of any one of embodiments 1 to 23, which is formulated for oral administration to a subject.
25. The composition of any one of embodiments 1 to 24, which is formulated for administration to a subject in doses between 0.01 g/kg body weight and 1 g/kg body weight.
26. The composition of embodiment 25 which is formulated for administration to a subject once, twice, three times, four times or several times per day.
27. The composition of any one of embodiments 1 to 26, which, when administered to a subject, leads to an increase of the ketone body concentration in the blood plasma.
28. The composition of embodiment 27, wherein the increase of the ketone body concentration in the blood plasma is physiologically effective.
29. The composition of any one of embodiments 25 to 28, wherein the ketone body concentration in the blood plasma is maintained for a prolonged period of time within the desired range.
30. The composition of embodiment 29, wherein the prolonged period of time is about 2, 4, 6, 8, 10, 12 or 14 hours.
31. The composition of embodiment 30, wherein the prolonged period of time is 7 to 8 hours.
32. The composition of embodiment 31, wherein the prolonged period of time is about 8 hours.
33. The composition of embodiment 30, wherein the prolonged period of time is 11 to 14 hours.
34. The composition of embodiment 33, wherein the prolonged period of time is about 12 hours.
35. The composition of any one of embodiments 27 to 34, wherein the ketone body concentration in the blood plasma is between about 0.3 mM and about 5 mM.
36. The composition of embodiment 35, wherein the concentration is between 0.8 mM and about 2 mM.
37. The composition of any one of embodiments 1 to 36 for use as a medicament.
38. The composition of any one of embodiments 1 to 36 for use as a medical food and/or food for special medical purposes.
39. The composition of any one of embodiments 1 to 38 for use in the treatment of neurological diseases.
40. The composition of embodiment 39, wherein at least one neurological disease is selected from epilepsy, migraine, autism, depression, affective disorders, anxiety and traumatic brain injury.
41. The composition of any one of embodiments 39 to 40, wherein the neurological diseases are epilepsy and/or migraine.
42. The composition of any one of embodiments 1 to 41 for use in the treatment of neurodegenerative diseases.
43. The composition of embodiment 42, wherein at least one neurodegenerative disease is selected from Alzheimer's disease, Parkinson's disease and multiple sclerosis.
44. The composition of any one of embodiments 42 to 43, wherein the neurodegenerative disease is Alzheimer' disease.
45. The composition of any one of embodiments 1 to 38 for use in the treatment of metabolic disorders.
46. The composition of embodiment 45, wherein at least one metabolic disorder is selected from glycogen storage disease, Acyl-CoA dehydrogenase deficiency and GLUT1 deficiency syndrome.
47. The composition of any one of embodiments 1 to 38 for use in the treatment of cancer.
48. A food product comprising the composition of any one of embodiments 1 to 36.
49. A food product of embodiment 48 to increase cognitive performance, decrease food craving, decrease body weight, decrease the fat to body weight ratio, maintain or improve muscle power, and/or improve athletic performance and/or endurance of a subject.
50. A food supplement comprising the composition of any one of embodiments 1 to 36.
51. A food supplement of embodiment 50 to increase cognitive performance, decrease food craving, decrease body weight, decrease the fat to body weight ratio, maintain or improve muscle power, and/or improve athletic performance and/or endurance of a subject.
52. A method to produce a composition of any one of embodiments 1 to 36.
53. A method of embodiment 52 comprising the steps of
   (a) preparing a denatured protein solution
   (b) combining a ketone body defined in any one of embodiments 2 to 10 with the protein solution of step (a)
   (c) extruding the mix of step (b) through an orifice forming microdroplets that free-fall in into a polymerization bath
   (d) curing the microbeads formed by steps (a-c)
   (e) drying of the microbeads of step (d)
54. A method of any one of embodiments 52 or 53 wherein the ketone body is R-3-hydroxybutyric acid and/or a pharmaceutically acceptable salt of R-3-hydroxybutryrate and said ketone body is combined with a 10% w/v denatured pea protein solution before extrusion through an orifice.

### BRIEF DESCRIPTION OF THE FIGURES

FIG. 1. Macronutrient content of participants' diets 2 days prior ingestion of βHB, expressed as g per kg body mass. CHO: carbohydrates; PRO: protein.

FIG. 2. Kinetics of βHB blood concentration upon ingestion of a dose of βHB. Data are mean±SD for n=8.

FIG. 3. Kinetics of glucose blood concentration upon ingestion of a dose of βHB. Data are mean±SD for n=8.

FIG. 4. Urine specific gravity in response to ingestion of a dose of βHB.

FIG. 5. Urine osmolality in response to ingestion of a dose of βHB.

FIG. 6. Macronutrient content of participants' diets 2 days prior ingestion of βHB, expressed as g per kg body mass. CHO: carbohydrates; PRO: protein. The data for the participants of the second trial (see Example 3) are shown.

FIG. 7. Kinetics of βHB blood concentration upon ingestion of 18 g βHB contained in microbeads, 18 g free βHB, or empty microbeads (see Example 3). Data are mean±SD for n=10.

FIG. 8. Kinetics of acetoacetate (AcAc) blood concentration upon ingestion of 18 g βHB contained in microbeads, 18 g free βHB, or empty microbeads (see Example 3). Data are mean±SD for n=10.

FIG. 9. Kinetics of glucose blood concentration upon ingestion of 18 g βHB contained in microbeads, 18 g free βHB, or empty microbeads (see Example 3). Data are mean±SD for n=10. A snack was provided at 6 h.

### DEFINITIONS

### Compound

A compound, as used herein, is an essentially pure chemical substance which however may comprise different enantiomers.

### Use of Singular and Plural

A ketone body, as used herein as singular, is a compound and not a single molecule.

Ketone bodies, as used herein as plural, refer to different compounds whereof each is a ketone body.

A microbead as used herein in its singular form, is an entity composed of many similar, but no necessarily identical, particles generated by the same production process.

Microbeads, as used herein as plural, refer to different entities whereof each entity is a different microbead.

This use of singular and plural extends to any other substance whereof it is obvious to a person skilled in the art that in the context it is used, the singular refers to a compound/entity regardless of the amount of molecules or particles and the plural refers to different compounds/entities. However, generally, terms in singular can comprise a plural meaning when it is clear from the context, and vice versa.

### Subject

The term subject, as used herein, refers to a human or an animal. In particular, a subject is a mammal, preferably a human.

### Ketone Body

A ketone body (KB), as used herein, refers to an endogenous ketone body or a precursor of an endogenous ketone body and/or a molecule containing one or more endogenous ketone body moieties. The term “ketone body” may further include a pharmaceutically acceptable salt of a ketone body as defined above.

A precursor of an endogenous ketone body produces an endogenous ketone body upon administration to a subject or during preparation of said precursor for administration to a subject. Said precursor may be a metabolic precursor of an endogenous ketone body.

An endogenous ketone body, as used herein, includes βHB and AcAc. Said endogenous ketone bodies, especially D-βHB and AcAc, are produced by the liver from fatty acids released from adipose tissue in times of starvation, fasting, glucose and/or carbohydrate deprivation and/or prolonged intense exercise. They can be used as an alternative energy substrate to glucose by most tissues of the body, most notably the brain, which cannot metabolize any other energy substrate apart from glucose and ketone bodies.

βHB, as used herein, refers to any enantiomer and/or racemate of beta-hydroxybutyric acid, also known as β-hydroxybutyric acid or 3-hydroxybutyric acid and/or its conjugate base beta-hydroxybutyrate, also known as β-hydroxybutyrate or 3-hydroxybutyrate (CAS No. 300-85-6).

D-βHB, also known as (R)-βHB, as used herein, refers to a specific enantiomer of βHB. Said enantiomer can be produced in the liver by humans.

AcAc refers to acetoacetic acid and/or its conjugate base acetoacetate (CAS No. 541-50-4).

An endogenous ketone body moiety may be a hydroxybutyrate moiety or an acetoacetyl moiety.

An exogenous ketone body, as used herein, refers to any ketone body that is not produced within a subject, but is delivered to said subject. It may be chemically identical to an endogenous ketone body.

A pharmaceutically acceptable salt, as used herein, may be selected from, but not to limited to, a sodium salt, a calcium salt, a magnesium salt, an arginine salt, a lysine salt, a leucine salt, a histidine salt, an ornithine salt, a creatine salt, an agmatine salt, a citrulline salt, a methyl glucamine salt and a carnitine salt, or a combination of said salts. A preferred combination is a combination comprising a calcium, a sodium, a potassium, a magnesium salt, in particular, a combination of a calcium, a sodium, a potassium, a magnesium and a lysine salt.

### Microbead

The terms “microbead”, “microcapsule”, “microencapsulate”, “microparticle”, “microparticulate” and “microsphere” are used interchangeably herein.

Microbeads, as used herein, refer to spherical particles with an average diameter of 50 to 500 microns, wherein the term “spherical” does not refer to a sphere in a strict sense, but refers to an object resembling a sphere from outside. Such an object may have any number of dints and bulges but resembles in average from outside more a sphere or an ellipsoid than for example, a cube, a pyramid, a cylinder or a filament. Preferably, microbeads have an average diameter of 50-200 microns, 80-200 microns or 80-150 microns.

The microbeads of the present invention comprise a pharmaceutically acceptable matrix. A pharmaceutically acceptable matrix, as used herein, may comprise for example, but not limited to, polymerized denatured pea protein, in particular yellow pea protein, soy bean protein, whey protein, gelatin, casein, or EUDRAGIT® polymers. EUDRAGIT® polymers are copolymers derived from esters of acrylic and methacrylic acid whose physicochemical properties are determined by functional groups. A pharmaceutically acceptable matrix may further comprise any material which is commonly used for the preparation of tablets or capsules to enclose the active part of a drug.

The term “microbead” should be understood as a microbead containing at least one ketone body, where it is clear to a skilled person from context, that a ketone body is comprised in the microbead. Such a context is for example, but not limited to, the application of a microbead to achieve a biological effect related to a ketone body and/or an effect of a ketone body, or a microbead during the production process where it is clear that a ketone body has already been enclosed.

One purpose of enclosing a ketone body within a microbead is to prevent undesired alterations of the physical or chemical properties of said ketone body by the environment and/or prevent said ketone body to affect the environment in an undesired way. The environment of a ketone body may be, but not limited to, the atmosphere, which plays a role for storage, a liquid wherein a ketone body is solved or suspended for administration to a subject or the gastrointestinal tract of a subject. In other words, englobbing or encapsulation may be used, for example, to reduce the hygroscopy of a ketone body, to increase the stability of a ketone body in an aqueous solution and/or to prevent its degradation and/or uptake in an undesired location such as the stomach. Englobbing or encapsulation may be further used to prevent a ketone body from eliciting an adverse effect in a subject upon intake, for example, the sensation of a bad taste and/or gastrointestinal distress.

The terms “englobbing” and “encapsulation” are used herein interchangeably and refer to the enclosure of a ketone body in a microbead.

A microbead particle containing a ketone body may have a variety of structures and shapes. A ketone body comprised in a microbead may be dispersed in the matrix, for example, homogeneously dispersed in the matrix, enriched in one or more locations, or separated from the matrix of a particle of said microbead. In a preferred embodiment, said microbead particles are multinuclear beads, wherein a ketone body is enriched in pockets (“nucleus”) which are dispersed throughout the matrix.

The term “dispersed” means that a ketone body molecule or a pocket of ketone body molecules, does not have a specific location in a microbead, but is distributed throughout a microbead. A “pocket” or “nucleus” enriched for ketone body molecules may be separated from the matrix to a variable extent. If a pocket is clearly separated from the matrix, the pocket rather refers to a “core”, and the matrix to a “shell” of a microbead.

In certain embodiments, a ketone body is contained in one or more “cores” of the microcapsule particle, and the matrix, i.e. the pharmaceutically acceptable matrix, as described herein, is contained in one or more “shells” or essentially constitutes one or more “shells” of said microbead particle. A shell, but not a core, can essentially constitute the outer surface of a microcapsule particle. A microcapsule particle may contain one or more core and/or shell layers. Cores and shells may be located at any location of the microcapsule particle which is not the outer surface. A microbead particle may be an intermediate of any of above described structures. A microbead may comprise microbead particles with various structures.

The term “microbead” may sometimes refer to a microbead particle as a person skilled in the art can infer from the context. For example, the shape of a microbead refers to the shape of a microbead particle.

A microdroplet refers to an intermediate during the production of a microbead. In particular, a microdroplet is formed when a suspension is extruded through an orifice or nozzle, preferably by using a vibrating nozzle technique.

A polymerization bath, as used herein, refers to a liquid wherein a microdroplet falls upon extrusion through an orifice or nozzle and in which the protein comprised in said microdroplet polymerizes as a consequence. In particular, a polymerization bath used for the gelling/polymerization of yellow pea protein comprised in a microdroplet, is acidic. Preferably, the polymerization bath comprises citrate.

The terms “polymerization” and “gelling” are used herein interchangeably and refer to a process, wherein smaller units bind to each other to form larger units. Preferably, this process is a chemical reaction. For example, several monomers and/or oligomers may react with each other to form a polymer. A polymer may be a gel or be comprised in a gel.

### Physiologically Effective

As used herein, the blood concentration of an active substance, i.e. a ketone body according to the invention, is considered to be physiologically effective when it has a desired and measurable biological effect and/or it is considerably higher (at least 1.8 fold, preferably at least 2.4 fold, preferably at least 3-fold higher) than the baseline concentration. Evidently, the physiologically effective concentration has an upper limit and does not exceed, at least not for more than 1, 2 or 4 hours, the maximum tolerated concentration. Thus, the blood concentration of a ketone body may be, in particular, at most 100-fold or 50-fold, preferably at most 20-fold, 10-fold or 5-fold increased over the baseline concentration. As known in the art, a desired and measurable biological effect of a ketone body can occur within a broad range, for example between about 0.3 mM and about 5 mM, in particular between 0.5 mM and 3 mM, preferably between 1 mM and 3 mM or between 0.8 mM and 2 mM. Thus, the ketone body concentration may be increased and/or maintained at said concentration(s) by ingesting the inventive composition and/or microbead provided herein. The baseline concentration is the concentration measured before ingestion of a ketone body or shortly thereafter. “Shortly thereafter” refers to a time period where the ketone body blood concentration is not significantly increased compared to before ingestion.

An active substance may be any substance which can have a biological effect. In the context used herein, an active substance refers to a ketone body.

In certain embodiments, a concentration is considered to be physiologically effective when the concentration is at least 1.8-fold higher compared to baseline concentration, even when no biological effect is determined.

In certain embodiments, a concentration is considered to be physiologically effective when a beneficial effect in a subject suffering from a disorder or disease is apparent upon one, multiple or regular ingestion(s) of a composition of the present invention.

The terms “therapeutically effective” or “therapeutically relevant” may be used herein in replacement of “physiologically effective”.

The term “therapeutic ketosis” refers to a physiologically effective ketone body blood concentration. In particular, the concentration is between 1 mM and 3 mM, preferably wherein the ketone body is βHB, more specifically D-βHB.

The terms “blood”, “plasma” and “blood plasma” are interchangeably used herein and refer to the blood plasma.

### Sustained or Controlled Release Dosage

A sustained release dosage and a controlled release dosage are variants of an extended release dosage. Administration of an active substance, herein a ketone body, or a composition containing said active substance, i.e. an inventive composition and/or microbead provided herein, to a subject in a sustained or controlled release dosage, as used herein, allows maintaining the blood concentration of said active substance in said subject at a physiologically effective level for a prolonged period of time. In a sustained release dosage, the active substance may be released at different rates over a prolonged period of time, whereas in a controlled release dosage said release rate may be nearly constant. As used herein, there is no sharp distinction between a sustained and a controlled release dosage, but the terms rather refer to gradual differences, wherein a controlled release dosage leads to a more stable ketone body blood concentration over time than a sustained release dosage. In particular, a controlled release dosage, as used herein, allows maintaining a ketone body blood concentration within a desired range for a prolonged period of time.

The desired range means that the concentration of said active substance in the blood may fluctuate around the desired physiologically effective concentration, but it does not go below the minimum effective concentration (MEC) or above the maximum tolerated concentration (MTC) for a time period which is considered by a person skilled in the art to be relevant. For example, a relevant time period is 4 hours, 2 hours or 1 hour and refers to the prolonged period of time, where the ketone body concentration is generally increased except during said relevant time period. Said fluctuations include any deviations from the desired concentration within the MEC and MTC. Preferably, fluctuations are increases and decreases of the concentration without a specific pattern.

The minimum effective concentration, as used herein, is defined by the minimum blood concentration of an active substance at which said substance is physiologically effective as described herein.

The maximum tolerated concentration, as used herein, is defined by the maximum blood concentration of an active substance at which said substance has no unacceptable toxicity as judged by a person skilled in the art and/or as described herein.

The desired concentration, the desired range, the MEC and/or the MTC may depend on the active substance, the subject to which the substance is delivered and/or the intended use of said substance.

Maintenance of the blood concentration of an active substance for a prolonged period of time, as used herein, means that the blood concentration of said substance is maintained longer than a person skilled in the art would expect for administration of said substance at the same dose in an immediate release dosage. An example of the βHB blood plasma kinetics upon ingestion of an immediate release dosage formulation (without microencapsulation) of βHB can be found in WO2018115158A1. As illustrated in Example 3, a prolonged period of time refers preferably to a prolongation of at least 3 hours, preferably at least 6 hours, preferably at least 8 hours, and/or an at least 2-fold, preferably 3-fold longer time for which the ketone body blood concentration is maintained. The term “maintenance” is defined by the desired range as explained above. The terms “prolonged period of time” and “extended time period” and grammatical variations thereof are used interchangeably herein.

### Suspension

The term “suspension”, as used herein, includes a colloidal suspension and refers to a mixture, wherein solid particles are dispersed in a liquid phase. In, particular, solid particles are microbead particles and the liquid phase is an aqueous solution.

### Disorder/Disease

The terms “disorder”, “disease” and “medical condition” are used herein interchangeably and refer to a pathophysiological response to external or internal factors, a disruption of normal or regular functions in the body or a part of the body and/or an abnormal state of health that interferes with the usual activities or feeling of wellbeing.

### Treatment

As used herein, “treatment” (and grammatical variations thereof such as “treat” or “treating”) refers to clinical intervention in an attempt to alter the natural course of the individual being treated. Desirable effects of treatment include, but are not limited to, prophylaxis, preventing occurrence or recurrence of disease or symptoms associated with disease, alleviation of symptoms, diminishment of any direct or indirect pathological consequences of the disease, decreasing the rate of disease progression, amelioration or palliation of the disease state, improved prognosis and cure.

### Medicament

The term “medicament”, as used herein, refers to a drug to cure, treat, prevent and/or diagnose a disease. In particular, a medicament is used for the treatment of a disease as described above.

### Food for Special Medical Purposes

A food for special medical purposes, as used herein, refers to food products for the dietary management (under medical supervision), of individuals who suffer from certain diseases, disorders or medical conditions. These foods are intended for the exclusive or partial feeding of people whose nutritional requirements cannot be met by normal foods.

### Medical Food

A medical food, as used herein, refers to a food which is formulated to be consumed or administered enterally under the supervision of a physician and which is intended for the specific dietary management of a disease or condition for which distinctive nutritional requirements, based on recognized scientific principles, are established by medical evaluation.

### Food Product

The terms “food” and “food product” are used interchangeably herein and refer to any substance consumed to provide nutritional support for an organism. Food may be of biological origin, and contain essential nutrients, such as carbohydrates, fats, proteins, vitamins, or minerals and/or consist essentially of an aqueous solution. Food is ingested by a subject and assimilated by the subject's cells to provide energy, maintain life, or stimulate growth. A food product can be taken in by a subject or administered to a subject. Preferably, a food product is eaten or drunk.

### Food Supplement

A food supplement, as used herein, refers to a concentrated source of nutrients with a nutritional and/or physiological effect. In the present invention, the main nutrient comprised in a food supplement is a composition comprising one or more ketone bodies contained in a microbead. A food supplement may be formulated for administration in a certain dosage, in particular, a sustained or controlled release dosage and/or a certain dose, in particular, to achieve a desired physiological effect in a subject. A food supplement may further comprise other nutrients, in particular vitamins or minerals. A food supplement can be taken in by a subject or administered to a subject. Preferably, a food supplement is eaten or drunk.

### EXAMPLES

The following are examples of methods and compositions of the invention. It is understood that various other embodiments may be practiced, given the general description provided above.

### Example 1

### Production of a Microbead Comprising βHB and a Matrix Essentially Consisting of Pea Protein

This example describes a process which revealed that a ketone body can be surprisingly enclosed in a microbead, and, more surprisingly, that the ketone body and/or a pharmaceutically acceptable salt thereof can constitute about 80-90% of the weight of the composition produced with in this process. The process included the following steps:

Step 1
A pea protein solution in aqueous 0.1 M NaOH to a concentration of 10% w/v (i.e 10 g/100 ml) was prepared. Preferably, the pea protein was yellow pea protein and fulfilled the standards for use in a drug.
It was ensured that the pH was in a range between 8 and 11.
The protein was solubilized by keeping the solution for at least 45 min at room temperature. It was ensured that the protein was well solubilized. The pH was adjusted to 7.5-10 using HCl or NaOH/KOH as required.
The solution was heat-treated at a temperature of at least 85° C. and maintained at that temperature for a duration of at least 25 min to denature the protein.

Step 2
The pea protein solution (10%) made according to Step 1 was combined with a βHB formulation in a volumetric ratio of approximately 1:9 to generate a dispersion. Preferably, the βHB formulation consisted of 50% R-3-hydroxybutyrate mineral salts (Na/Ca/K/Mg Blend 1:1:1:2) and 50% R-3-hydroxybutyric acid.
This dispersion was extruded through an orifice to form microdroplets that free-fall into a polymerization bath which comprised citrate at a concentration of 0.1-0.5 M. In particular, a vibrating nozzle technique was used, in which the suspension is sprayed (extruded) through a nozzle and laminar break-up of the sprayed jet is induced by applying a sinusoidal frequency with defined amplitude to the spray from the nozzle.
The microdroplets formed microparticulates which were allowed to cure in the polymerization buffer at low agitation speed for 2 hours at room temperature.

Step 3
The microparticulates were dried by using hot air circulation in a spray drier with an inlet temperature of 183° C. and an outlet temperature of 92° C.

The microbead particles produced in this process had an average diameter of about 50-200 microns, in particular, about 80-150 microns. In particular, the microbead particles comprised as βHB formulation a mix of R-3-hydroxybutyrate mineral salts (Na/Ca/K/Mg Blend 1:1:1:2) and R-3-hydroxybutyric acid which was enriched in pockets dispersed throughout a polymerized yellow pea protein matrix and wherein the βHB formulation constituted approximately 89 to 90% of the weight of the microbead.

### Example 2

### Maintenance of an Increased βHB Blood Concentration for a Prolonged Period of Time Upon Ingestion of a Microbead Comprising βHB

This example describes an interventional trial wherein the βHB blood concentration and further parameters were measured in subjects which ingested a dose of a composition comprising one or more ketone bodies contained in a microbead produced as described in example 1. Surprisingly, the βHB blood concentration was maintained at an increased level for a prolonged period of time upon ingestion of said composition comprising βHB as ketone body. Further measured parameters describe, for example, the tolerance, safety and/or efficacy of the composition.

### Composition and Dose

A dose was 15 g of a microbead containing a mix of 50% R-3-hydroxybutyrate mineral salts (Na/Ca/K/Mg Blend 1:1:1:2) and 50% R-3-hydroxybutyric acid (βHB formulation). In particular, said βHB formulation was enriched in pockets dispersed throughout a polymerized yellow pea protein matrix of the microbead particles and constituted approximately 89 to 90% of the weight of the microbead. Thus, the βHB formulation comprised in said dose was about 13.4 g. For administration, one dose of the composition was suspended in 250 ml of plain water.

### Participants

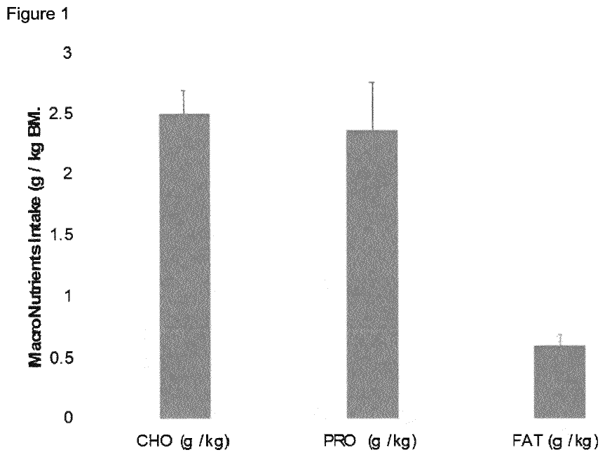

Four young males and four young females volunteered to participate in the study and were physically active as defined by habitually performing at least 30 min of exercise on five or more days each week. Age, height, body mass, BMI, body fat ratio, body fat and fat free mass were determined (Table 1). Height was measured using a wall-mounted stadiometer to the nearest 1 cm, and body mass using a digital scale to the nearest 0.1 kg. Each participant provided written informed consent prior to participation. Participants were asked to abstain from caffeine and alcohol and refrain from strenuous exercise for 24 h prior to the lab visit, and they were asked to keep a 2-day portion size estimate food diary prior to their visit (analysed using the Nutritics Software V1.15). The macronutrient content of participants' diet 2 days prior of the trial was in average about 2.5 g carbohydrates, 2.4 g protein and 0.6 g fat per kg body mass per day (FIG. 1).

TABLE 1
Assessment of trial participants

| Parameters | N = 8 |
|---|---|
| Age (y) | 24.0 ± 3.5 |
| Height (m) | 1.78 ± 0.22 |
| Body mass (kg) | 79.0 ± 5.3 |
| BMI (kg m⁻²) | 26.0 ± 1.4 |
| Percentage body fat (%) | 16.8 ± 1.9 |
| Body fat (kg) | 12.99 ± 2.02 |
| Fat free mass (kg) | 62.67 ± 3.44 |

### Indicators and Measured Variables

β-Hydroxbutyrate (βHB): in the blood
Glucose: in the blood
Specific gravity of Urine
Osmolality of Urine
Food Intake
Sensory Questionnaire

### Administration of the Composition and Sampling of Urine and Blood

Participants arrived at the laboratory between 7.30 and 9.00 after an overnight fast, and provided a urine sample for hydration assessment (PEN Refractometer, Atago Instruments). Participant parameters shown in Table 1 were determined. Next, a fasting blood sample was taken via a cannula inserted into a superficial forearm vein for serial blood sampling. After providing a “fasting” blood sample, participants ingested a 250 mL drink wherein the 15 g dose of a microbead comprising the βHB formulation as defined in the example herein was suspended in water. After supplementation, baseline determinations tests were repeated for comparison between the pre- and post-supplementation periods. Every 30 min for three hours after ingestion, a blood sample was taken. After 3 hours, a blood sample was taken at 1-hour intervals. After 8 hours, a blood sample was taken at 2-hour intervals. Participants remained in the lab at quiet rest throughout this time period. Participants were offered some small snack 6 hours after consumption of the prototype drink which they could choose from low caloric yoghurts, muesli bars and low caloric fruit juices. Most participants consumed yoghurts as their choice. Between samples the cannula was kept patent with isotonic saline (0.9% w/v sodium chloride, Baxter Healthcare). Blood samples (4.5 mL) were collected in plastic tubes containing lithium heparin (Vacuette, Greiner Bio-One) at each sampling point, including the “fasting” blood sample, followed by centrifugation for 10 min at 4000 rpm at 4° C. Plasma was stored at −80° C. until subsequent analysis. Urine samples were again taken 12 h post supplementation. The assisting researcher was not party to the subsequent blood analysis.

TABLE 2
Study schedule

| Activity | Timeline | |
|---|---|---|
| Arrival in Lab | −45 | min |
| Participant Assessment | −40 | min |
| Cannulation | −15 | min |
| “Fasting Blood” | 0 | h |
| Supplementation | 0 | h |
| Blood Draws 30 min Intervals | 0-3 | h |
| Blood Draws 1 h Intervals | 3-8 | h |
| Blood Draws 2 h Intervals | 8-12 | h |

### Determination of βHB and AcAc Levels in Blood Plasma Samples

βHB and AcAc standards, at two levels, were assayed per patient. The assay was considered to be correct if the standard values were within 1.1% of the assigned value. To calculate the analytical interval, the following standard aqueous solutions were used: 2500, 1250, 250, 100, 25, 5, 2.5, 1, 0.75, 0.5, 0.25, 0.1 mmol/L of βHB sodium salt, and 600, 500, 250, 100, 50, 25, 10, 25, 5, 2.5, 1, 0.75, 0.5, 0.25, 0.1 mmol/L acetoacetic acid salt. The detection limit (L_{d}) was established following the processing of 28 specimens of distilled water.

The recommendations of the European Committee for Clinical Laboratory Standards (ECCLS) were followed for the study of the imprecision and inaccuracy of the method. 0.1, 0.5, 1, 25, 50 and 100 mmol/L βHB and acetoacetate standard aqueous solutions were used. To determine the analytical recovery of the methods, increasing quantities of the βHB sodium salt or AcAc salt were added to aliquots of the pool of sera. The reference values of the methods were obtained and participant samples were evaluated.

### Determination of Glucose Levels in Blood Plasma Samples

Blood glucose levels were determined using High-performance liquid chromatography (HPLC).

### Effects of Ingestion of βHB Contained in a Microbead

### Increased βHB Plasma Levels for a Prolonged Period of Time

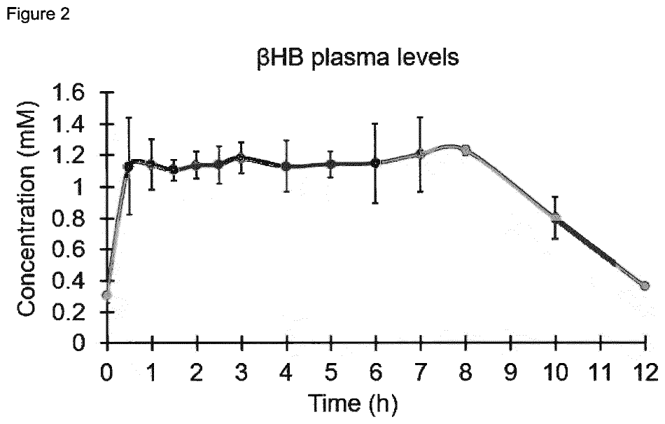

Average plasma βHB concentrations were between 1.0 and 1.3 mM at all sample time-points between 0.5 and 8 h after ingestion of the composition (FIG. 2). This represents an increase of about 3 to 5-fold over the baseline concentration. No consistent increase or decrease was observed between 0.5 and 8 h after ingestion. Plasma βHB levels started to decrease after 8 h but at 10 h the concentration was still elevated compared to baseline. In conclusion, ingestion of a composition comprising βHB contained in a microbead elevated βHB levels in the blood plasma to a therapeutically relevant concentration latest at 30 min upon intake and said concentration was stably maintained for about 7.5 h. In other words, the βHB plasma concentration was similarly elevated at 8 h after ingestion than at 0.5 h after ingestion of said composition. Furthermore, βHB levels in the blood plasma were elevated compared to baseline for another two hours, wherein the baseline refers to the concentration at the time-point of ingestion. It is surprising that βHB levels in the blood were maintained for such a long period of time at said concentration when compared to previous data showing that the βHB blood plasma concentration dropped after about 1 h upon ingestion of a 10 g dose of a βHB formulation; see WO2018115158A1.

### Inconspicuous Glucose Plasma Levels

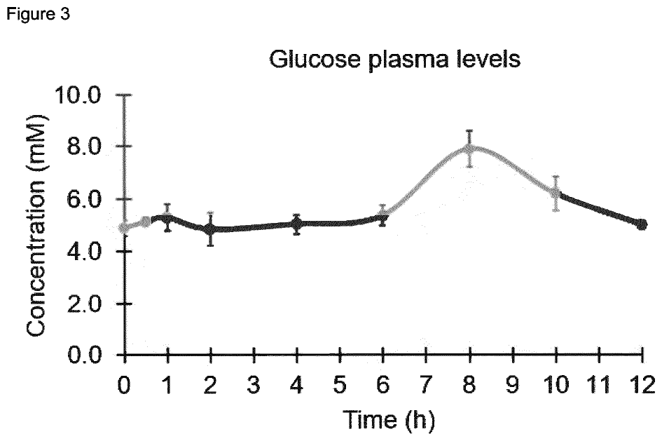

The blood glucose concentration over time was inconspicuous. Notably, blood glucose levels were not altered for 6 hours upon ingestion of the composition described in the example herein. Only, after 6 hours, when a snack was provided, blood glucose levels increased, but this would be also expected in subjects not having ingested said composition (FIG. 3). Thus, the ingestion of a dose described herein can be assumed to be safe regarding potentially dangerous low or high blood glucose levels.

### Unchanged Urine Gravity and Osmolality

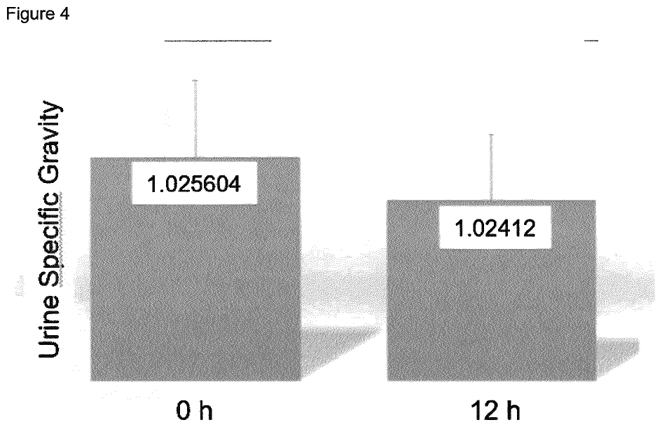

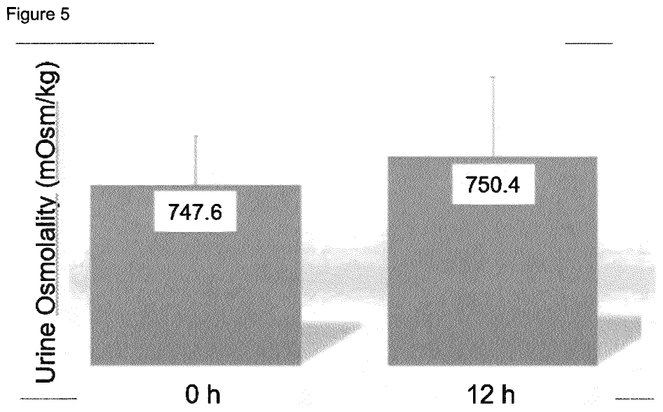

Assessment of the hydration of trial participants using the urine samples revealed no conspicuous changes (FIGS. 4 and 5). Thus, the ingestion of a dose described herein can be assumed to be safe regarding dehydration.

### Good Sensory Properties

Sensory evaluation shows that no significant dispersion issues were reported but a slight sweet perception in taste. No significant aroma issues were reported and no effect on alertness. Interestingly, a level of satiety was achieved for the majority of participants. 75% of the participants reported no GI discomfort (a frequent side-effect of supplementation with ketone bodies) and the remainder reported a slight to moderate discomfort. No extreme GI discomfort was reported. No participant disliked the composition and all participants reported a non-typical or neutral aroma showing that the composition had a good acceptance and no problem of taste.

TABLE 3
Sensory Questionnaire Feedback, N = 8

| | | |
|---|---|---|
| Appearance | Clear | 7 |
| | Cloudy | 1 |
| | Sediment | 0 |
| Aroma | Non-Typical | 5 |
| | Neutral | 3 |
| | Typical | 0 |
| Sweetness | Neutral | 2 |
| | Fairly Sweet | 5 |
| | Sweet | 1 |
| Texture | Floury, coarse mouthfeel | 0 |
| | Dry, chalky, mouthfeel | 3 |
| | No detected mouthfeel | 0 |
| | Sandy, fine mouthfeel | 2 |
| | Smooth, melts in the mouth | 3 |
| Aftertaste | Strong aftertaste | 2 |
| | Slight aftertaste | 3 |
| | Nothing detected/neutral | 3 |
| Fullness | Extreme fullness feeling | 0 |
| | Moderate fullness feeling | 4 |
| | Satisfied feeling | 3 |
| | Slightly Hungry | 1 |
| | Strong Hunger | 0 |
| GI Discomfort | Extreme Discomfort | 0 |
| | Moderate discomfort | 1 |
| | Slight discomfort | 1 |
| | No Effect | 6 |
| Acceptance | Dislike Extremely | 0 |
| | Dislike | 0 |
| | Neither Like nor Dislike | 6 |
| | Like | 1 |
| | Like Extremely | 1 |
| Alertness | Extreme Alertness | 0 |
| | Moderate Alertness | 0 |
| | Slight Alertness | 2 |
| | No effect | 6 |
| Awake | Extremely awake | 0 |
| | Awake with no drowsiness/ | 2 |
| | tiredness | |
| | No Effect | 6 |
| | Slightly more drowsiness | 0 |
| | than normal | |
| | Extremely drowsiness and | 0 |
| | tired | |
| Fitness | Extreme activity boost | 0 |
| | Slight activity boost | 2 |
| | No effect | 5 |
| | Lethargic/no energy | 1 |
| How many hours a day | Over 12 hours a day | 0 |
| are you eating/ | Between 12 and 10 hours a day | 0 |
| drinking caloric | Between 10 and 8 hours a day | 0 |
| beverages | Between 8 and 6 hours a day | 1 |
| | Between 6 and 4 hours a day | 6 |
| | Less than 4 hours a day (one | 1 |
| | meal only) | |
| How many meals a day | Over 1-2 | 0 |
| do you consume on | Over 3-4 | 3 |
| average (including | Over 5-6 | 5 |
| snacks) | Over 7 or more | 0 |
| How many times have | Once | 3 |
| you fasted in your life | Twice | 0 |
| (fasting = 24 hours/24 | Three | 0 |
| hour + without eating | Never | 5 |
| | +3 times | 0 |
| | If yes what was the reason: | Training/ |
| | | Sickness |

### Example 3

### Maintenance of an Increased βHB and AcAc Blood Concentration for a Prolonged Period of Time Upon Ingestion of a Microbead Comprising 18 g βHB

This example describes a further independent interventional trial with different participants than in Example 2, wherein the βHB blood concentration and further parameters, i.e. acetoacetate (AcAc) and glucose blood concentrations, were measured in subjects which ingested a dose of a composition comprising one or more ketone bodies contained in a microbead produced as described in example 1. Surprisingly, the βHB and AcAc blood concentrations were maintained at an increased level for a prolonged period of time, in particular for at least about 12 h, upon ingestion of said composition comprising βHB as ketone body. In particular, βHB enclosed in microbeads allowed to prolong the time a relevant ketone body concentration was maintained by at least 8 hours (to a total of at least about 12 hours) compared to free βHB (at most 3 hours). Thus, said maintenance time was at least 3-fold longer. Further measured parameters describe, for example, the tolerance, safety and/or efficacy of the composition. In contrast to Example 2, about 18 g of βHB contained in microbeads (versus about 13.4 g) were administered to the trial participants and the results were directly compared to the administration of non-enclosed (free) βHB and empty microbeads. Furthermore, plasma acetoacetate concentrations were additionally determined.

Moreover, the study was a single-center, blinded randomized cross-over study.

Each participant had three visits (and thus could be also used as his/her own control). The participants went to the visits A, B and C in a randomized order. At least 3 days have passed between each treatment.
Visit A: Oral ingestion of non-enclosed βHB
Visit B: Oral ingestion of βHB contained/enclosed in microbeads
Visit C: Oral ingestion of microbeads without any content (empty).

### Composition and Dose

A dose of βHB contained in microbeads was 20 g of microbeads containing a mix of 50% R-3-hydroxybutyrate mineral salts (Na/Ca/K/Mg Blend 1:1:1:2) and 50% R-3-hydroxybutyric acid (βHB formulation). In particular, said βHB formulation was enriched in pockets dispersed throughout a polymerized yellow pea protein matrix of the microbead particles and constituted approximately 89 to 90% of the weight of the microbead. Thus, the βHB formulation comprised in said dose was about 18 g, in particular about 17.8 g.

The dose of non-enclosed (free) βHB was also about 18 g, in particular about 17.8 g, and the dose of empty microbeads was about 2 g, in particular about 2.2 g. Thus, the free βHB and empty microbeads were direct controls of the composition comprising βHB contained in microbeads.

For administration, one dose was suspended in 250 ml of lemon-flavored non-caloric water.

### Participants

Four young males and six young females (age: 22-35 years; average: 27 years) volunteered to participate in the study. The participants were healthy and were not taking pharmaceutical drugs, only anticonceptives were allowed. In particular, the participants had no symptoms associated with gastrointestinal dysfunction, were not pregnant or breastfeeding, were not allergic against standard meals or treatment, have not abused substances, had no history of diabetes or migraine, had no experience with ketone bodies or a ketogenic diet, and were not professional athletes.

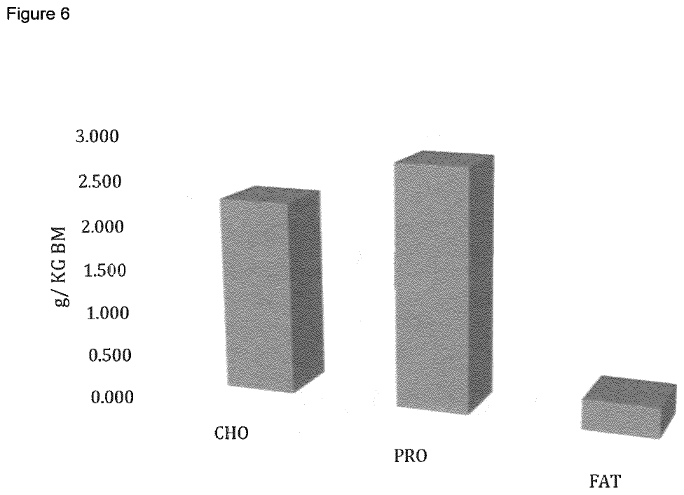

Each participant provided written informed consent prior to participation. Participants were asked to abstain from caffeine and alcohol and refrain from strenuous exercise for 24 h prior to the lab visit, and they were asked to keep a 2-day portion size estimate food diary prior to their visit (analysed using the Nutritics Software V1.15). The macronutrient content of participants' diet 2 days prior of the trial was in average about 2.0 g carbohydrates, 2.6 g protein and 0.1 g fat per kg body mass per day (FIG. 6).

### Indicators and Measured Variables

β-Hydroxbutyrate (βHB): in the blood
Acetoacetate (AcAc): in the blood
Glucose: in the blood
Osmolality of Urine
Food Intake
Sensory Questionnaire

### Administration of the Composition and Sampling of Urine and Blood

Participants arrived at the laboratory between 7.30 and 9.00 after an overnight fast, and provided a urine sample for hydration assessment (PEN Refractometer, Atago Instruments). Next, a fasting blood sample was taken via a cannula inserted into a superficial forearm vein for serial blood sampling. After providing a “fasting” blood sample, participants ingested a 250 mL lemon flavored non-caloric water drink wherein the 18 g dose of a microbead comprising the βHB formulation was suspended. After supplementation, baseline determinations tests were repeated for comparison between the pre- and post-supplementation periods. Every 30 min for three hours after ingestion, a blood sample was taken. After 3 hours, a blood sample was taken at 1-hour intervals. After 8 hours, a blood sample was taken at 2-hour intervals. Participants remained in the lab at quiet rest throughout this time period and were permitted to use entertainment headsets and the digital library and were allowed make short walks outside. Participants were offered some small snack 6 hours after consumption of the prototype drink which they could choose from low caloric yoghurts, muesli bars and low caloric fruit juices. Between samples the cannula was kept patent with isotonic saline (1% w/v sodium chloride, Baxter Healthcare). Blood samples (4.3 mL) were collected in plastic tubes containing lithium heparin (Vacuette, Greiner Bio-One) at each sampling point, including the “fasting” blood sample, followed by centrifugation for 10 min at 4000 rpm at 4° C. The blood samples were, at least for the determination of AcAc levels, directly analyzed within 2 hours (or otherwise stored at −80° C. before analysis). Urine samples were again taken 12 h post supplementation.

The Determination of βHB, AcAc and glucose levels in blood plasma samples was as described in Example 2.

### Effects of Ingestion of 18 g βHB in Either Free Form or Enclosed in a Microbead

The experimental setup allowed to directly determine the physiological effects of ingesting βHB enclosed in microbeads compared to standard non-enclosed βHB. To distinguish the effects of βHB from effects of the enclosing material, i.e. the pea protein, participants have also ingested empty microbeads on one trial day.

### Tolerability of βHB

No participant has reported nausea upon ingestion of microbeads, independent if said microbeads were empty or contained βHB. In contrast, within the first hour upon ingestion of free βHB, 7 of 10 participants reported gastrointestinal distress such as stomach cramping, upset, nausea, bloating and diarrhea. Specifically, 3 participants expressed severe discomfort and sickness, and further 4 participants expressed mild nausea. It took the effort of the clinical team to convince the participants to not quit the trial. This demonstrates that a relatively large dose of non-enclosed βHB was poorly tolerated whereas the same dose contained in the microbeads was well tolerated.

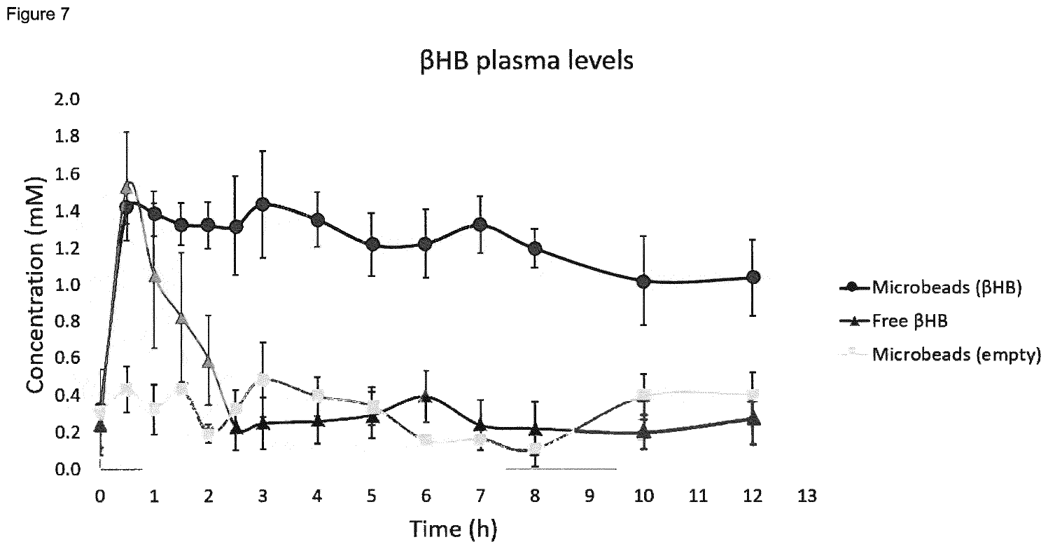

### βHB Plasma Levels (FIG. 7)

### a) Ingestion: Free βHB

The average plasma βHB concentration peaked at the first measurement time-point (0.5 h) where it was 1.5 mM. Within 2 hours, the plasma βHB concentration has dropped to baseline levels, where it stayed until the end of the experiment (12 h). Thus, a therapeutically relevant blood βHB concentration was only achieved for at most 2.5 h, probably for only about 2 h.

### b) Ingestion: Empty Microbeads

Ingestion of empty microbeads did not elevate the average plasma βHB concentration over baseline (0 h) during the experiments. This demonstrates that any effects of microbeads containing βHB on the plasma βHB concentration are due to βHB and not the enclosing material.

### c) Ingestion: Microbeads Containing βHB

Average plasma βHB concentrations were between 1.0 and 1.5 mM at all sample time-points between 0.5 and 12 h after ingestion of the composition. This represents an increase of about 3 to 5-fold over the baseline concentration. The peak concentration was at least 90% of that for free βHB but, in contrast to free βHB, a nearly as high concentration was maintained until the end of the experiment (12 h). Only a very slight (<30%) decrease was observed between 0.5 and 12 h after ingestion. However, the plasma βHB concentration after 12 h was still more than 3-fold increased compared to baseline (0 h). In conclusion, ingestion of a composition comprising βHB contained in a microbead elevated βHB levels in the blood plasma to a therapeutically relevant concentration latest at 30 min upon intake and said concentration was stably maintained for about 11.5 h. In other words, the βHB plasma concentration was similarly elevated at 12 h after ingestion than at 0.5 h after ingestion of said composition, at least in a therapeutic context. Regarding the steep slope of βHB plasma level increase within the first 0.5 h, it is reasonable to assume that the maintenance of an increased plasma βHB concentration was for at least about 12 h, and not only 11.5 h as directly proven by the data.

Thus, enclosing βHB in microbeads allowed to maintain a therapeutically relevant plasma βHB concentration for at least about 12 h, as compared to at most 2.5 h for free βHB. In other words, an increased, in particular therapeutically relevant, plasma βHB concentration was maintained at least about three times longer by enclosing βHB in microbeads.

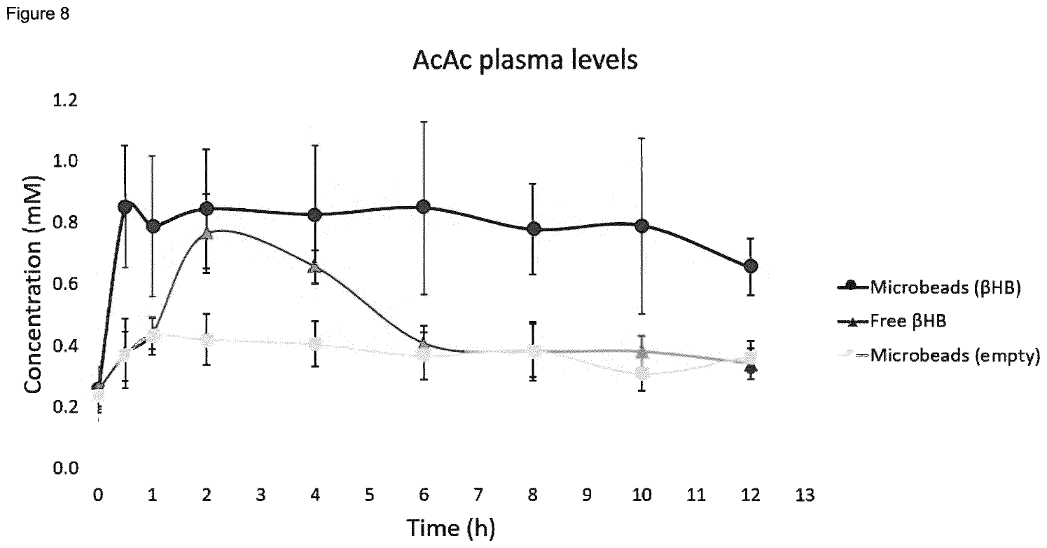

### Acetoacetate (AcAc) Plasma Levels (FIG. 8)

### a) Ingestion: Free βHB

The average plasma AcAc concentration peaked at 2 h where it was 0.8 mM. Within 4 hours, the plasma βHB concentration has dropped to baseline levels, where it stayed until the end of the experiment (12 h). Of note, the base line appeared to be slightly higher after 1 h (see b). A therapeutically relevant increase of the blood AcAc concentration was only achieved for at most 3 h, probably for only about 2 h.

### b) Ingestion: Empty Microbeads

Ingestion of empty microbeads had no clinically relevant effect on the AcAc blood concentration. The average plasma AcAc concentration was slightly increased after 1 h, upon which it stayed similar until the end of the experiment. This slight elevation, however, was likely an artefact of fasting until the afternoon, rather than related to the 2 g of pea protein. Any additional effect of microbeads containing βHB is due to the βHB and not the enclosing material.

### c) Ingestion: Microbeads Containing βHB

Average plasma AcAc concentrations were between 0.6 and 0.9 mM at all sample time-points between 0.5 and 12 h after ingestion of the composition. This represents an increase of about 1.5 to 3-fold over the baseline concentration. The peak AcAc concentration was about 10% higher than that for free βHB and, in contrast to free βHB, a nearly as high concentration was maintained until the end of the experiment (12 h). Only a very slight decrease (<30%) was observed between 0.5 and 12 h after ingestion. However, the plasma AcAc concentration after 12 h was still more than 2-fold increased compared to baseline (0 h), or 1.8-fold compared to the empty microbeads at 12 h. In conclusion, ingestion of a composition comprising βHB contained in a microbead elevated AcAc levels in the blood plasma to a therapeutically relevant concentration latest at 30 min upon intake and said concentration was stably maintained for about 11.5 h. In other words, the AcAc plasma concentration was similarly elevated at 12 h after ingestion than at 0.5 h after ingestion of said composition, at least in a therapeutic context. Regarding the steep slope of AcAc plasma level increase within the first 0.5 h, it is reasonable to assume that the maintenance of an increased plasma AcAc concentration was for at least about 12 h, and not only 11.5 h as directly proven by the data.

Thus, enclosing βHB in microbeads allowed to maintain a therapeutically relevant plasma AcAc concentration for at least about 12 h, as compared to at most 3 h for free βHB. In other words, an increased, therapeutically relevant, plasma AcAc concentration was maintained at least about three times longer by enclosing βHB in microbeads.

It was surprising that the ingestion of βHB contained in microbeads not only increased and maintained βHB levels in the blood at a therapeutically relevant concentration for at least about 12 h but increased and maintained also AcAc levels in the blood in the same way.

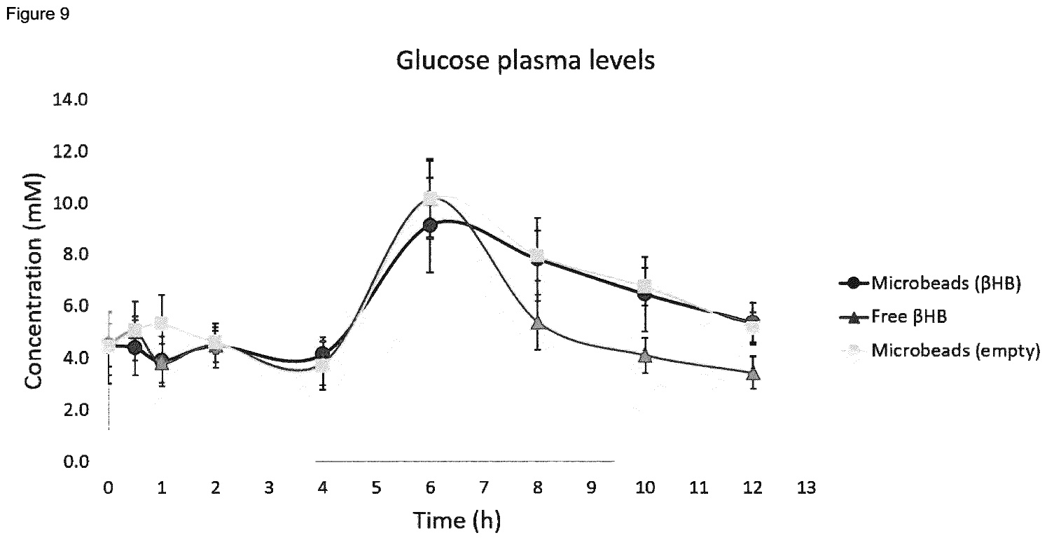

### Glucose Plasma Levels (FIG. 9)

The blood glucose concentration was largely similar over time for a) free βHB, b) empty microbeads, and c) microbeads containing βHB. Notably, blood glucose levels were not altered for at least 4 hours upon ingestion of the sample. However, after 6 hours, when a snack was provided, blood glucose levels increased as expected. Since no significant differences to empty microbeads were observed, it is safe to assume that ingestion of a dose of microbeads containing βHB as described herein does not lead to potentially dangerous low or high blood glucose levels.

### Unchanged Urine Osmolality

Assessment of the hydration of trial participants using urine samples revealed no apparent change in urine osmolality post supplementation for any condition. Thus, the ingestion of any of the doses described herein can be assumed to be safe regarding dehydration.

### Sensory Properties of Microbeads Containing βHB (Table 4)

Sensory evaluation shows that no significant dispersion issues were reported but a slight sweet perception in taste and some aftertaste. No significant aroma issues were reported and no effect on alertness or fitness. Interestingly, a level of satiety or moderate fullness was achieved for 80% of the participants. 80% of the participants reported no GI discomfort (a frequent side-effect of supplementation with ketone bodies as also observed in this study for free βHB) and the remainder reported only a slight discomfort. No moderate or extreme GI discomfort was reported. Only 30% of the participants disliked the composition (due to the texture) and all participants reported a non-typical or neutral aroma showing that the composition had an overall good acceptance and no problem of taste.

TABLE 4
Sensory Questionnaire Feedback 2, N = 10

| | | |
|---|---|---|
| Appearance | Clear | 1 |
| | Cloudy | 7 |
| | Sediment | 2 |
| Aroma | Non-Typical | 4 |
| | Neutral | 6 |
| | Typical | 0 |
| Sweetness | Neutral | 2 |
| | Fairly Sweet | 2 |
| | Sweet | 6 |
| Texture | Floury, coarse mouthfeel | 1 |
| | Dry, chalky, mouthfeel | 3 |
| | No detected mouthfeel | 0 |
| | Sandy, fine mouthfeel | 3 |
| | Smooth, melts in the mouth | 3 |
| Aftertaste | Strong aftertaste | 5 |
| | Slight aftertaste | 5 |
| | Nothing detected/neutral | 0 |
| Fullness | Extreme fullness feeling | 0 |
| | Moderate fullness feeling | 2 |
| | Satisfied feeling | 6 |
| | Slightly Hungry | 2 |
| | Strong Hunger | 0 |
| GI Discomfort | Extreme Discomfort | 0 |
| | Moderate discomfort | 0 |
| | Slight discomfort | 2 |
| | No Effect | 8 |
| Acceptance | Dislike Extremely | 0 |
| | Dislike | 3 (Texture) |
| | Neither Like nor Dislike | 7 |
| | Like | 0 |
| | Like Extremely | 0 |
| Alertness | Extreme alertness | 0 |
| | Moderate alertness | 0 |
| | Slight Alertness | 0 |
| | No effect | 10 |
| Awake | Extremely awake | 0 |
| | Awake with no drowsiness/ | 0 |
| | tiredness | |
| | No Effect | 10 |
| | Slightly more drowsiness | 0 |
| | than normal | |
| | Extremely drowsiness and | 0 |
| | tired | |
| Fitness | Extreme activity boost | 0 |
| | Slight activity boost | 0 |
| | No effect | 10 |
| | Lethargic/no energy | 0 |
| How many hours a day | Over 12 hours a day | 0 |
| are you eating/ | Between 12 and 10 hours a day | 0 |
| drinking caloric | Between 10 and 8 hours a day | 0 |
| beverages | Between 8 and 6 hours a day | 3 |
| | Between 6 and 4 hours a day | 6 |
| | Less than 4 hours a day (one | 1 |
| | meal only) | |
| How many meals a day | Over 1-2 | 0 |
| do you consume on | Over 3-4 | 2 |
| average (including | Over 5-6 | 8 |
| snacks) | Over 7 or more | 0 |
| How many times have | Once | 0 |
| you fasted in your life | Twice | 3 |
| (fasting = 24 hours/24 | Three times | 1 |
| hours + without eating) | Never | 6 |
| | +3 times | 0 |
| | If yes what was the reason: | Training/ |
| | | Sickness |

### Example 4

### Optimization of Enclosure of β-hydroxybutyric Acid (Free Acid βHB) into Microbeads

The physical and/or chemical stability of free acid βHB may be optimized using food grade silica. In particular, interactions between β-hydroxybutyric acid and water molecules may be minimized by adding silica (silicon dioxide) at a concentration of at most 4% (w/w), e.g. 3.2% to 3.4% (w/w). Moreover, moisture migration kinetics may be measured as a function of time and/or temperature, i.e. to ensure homogeneity between batches. For example, the attraction of water may be estimated by weighing the β-hydroxybutyric acid, and if the weight increases this may indicate absorption or adsorption of water molecules from the surrounding environment.

Furthermore, the enclosure efficiency and yield may be validated and compared to the microbeads produced as described in Example 1 without silica. Moreover, the moisture content of the produced microbeads may be determined and is ideally below 5%.

Moreover, the production parameters, e.g. the amount of free acid βHB, may be adjusted to ensure a similar controlled release functionality as demonstrated in Examples 2 and 3. In particular, a clinical trial may be carried out as described in Examples 2 and 3, and/or an in vitro digestion assay, as described in Example 5, may be performed to verify the controlled release functionality.

Further quality checks, i.e. regarding stability and purity may be performed as described in Example 5.

### Example 5

### In Vitro Validation of Microbeads and Quality Control

### Digestion Assay

An in vitro digestion assay may be performed, e.g. according to the INFOGEST method (Minekus (2014), Food Funct. 5(6):1113-24). In such an assay, the stability of the microbeads through the salivary phase, the gastric phase and the intestinal phase may be determined, and the release kinetics be defined as function of time and/or gastrointestinal section, thereby verifying the resistance against oral mechanical stress (20 min), the protection against gastric conditions (90 min), and the controlled intestinal release of βHB (12 hour).

### Storage Stability

Microbead's stability may be determined after storage in different conditions:
Water Activity (Aw) of microbeads containing βHB over 8 weeks at 30° C. and uncontrolled humidity
Moisture content of microbeads containing βHB for 4 weeks at 30° C. and uncontrolled humidity

### Purity

The purity of a ketone body formulation (i.e. D-βHB) to be enclosed may be determined by HPLC, UPLC-PDA and/or Fourier Transform InfraRed spectroscopy, i.e. by comparing with a known concentration of a reference standard of said ketone body (i.e. D-βHB).

1-72. (canceled)

73. A composition comprising one or more ketone bodies contained in a microbead.

74. The composition of claim 73, which, when administered to a subject, leads to an increase of the ketone body concentration in the blood plasma, wherein said ketone body concentration is at least 1.8-fold of a baseline concentration.

75. The composition of claim 74, wherein the ketone body concentration in the blood plasma is maintained for about 2, 4, 6, 8, 10, 12 or 14 hours within a desired range.

76. The composition of claim 74, wherein the ketone body concentration in the blood plasma is between about 0.3 mM and about 5 mM.

77. The composition of claim 73, wherein the ketone body concentration in the blood plasma is maintained for 2, 4, 6, 8, 10, 12 or 14 hours between about 0.3 mM and about 5 mM, when said composition is administered to a subject.

78. The composition of claim 77, wherein the ketone body concentration in the blood plasma is the blood plasma concentration of beta-hydroxybutyrate (bHB).

79. The composition of claim 73, wherein the microbead comprises a pharmaceutically acceptable matrix comprising polymerized protein.

80. The composition of claim 79, wherein the protein comprises denatured animal or vegetable protein.

81. The composition of claim 73, wherein the microbead does not consist of more than 90%, 70%, 50%, 30%, 10% or 0% poly-3-hydroxybutyrate and/or one or more other polymerized ketone bodies in volume and/or weight.

82. The composition of claim 79, wherein the one or more ketone bodies is/are dispersed throughout the pharmaceutically acceptable matrix comprised in the microbead.

83. The composition of claim 73, wherein the one or more ketone bodies and/or a pharmaceutically acceptable salt thereof, constitute(s) at least about 50%, 70%, 85% or 90% of the weight of the microbead.

84. The composition of claim 73, wherein the one or more ketone bodies contained in the microbead is/are selected from
(a) beta-hydroxybutyric acid or beta-hydroxybutyrate (bHB),
(b) acetoacetate (AcAc),
(c) a precursor of bHB and/or AcAc,
(d) a compound comprising an acetoacetyl- and/or 3-hydroxybutyrate moiety, and
(e) a pharmaceutically acceptable salt of any one of (a) to (d).

85. The composition of claim 73, wherein the one or more ketone bodies contained in the microbead comprises or is bHB and/or a pharmaceutically acceptable salt thereof.

86. The composition of claim 73, wherein the one or more ketone bodies comprises a mix of R-3-hydroxybutyric acid and a pharmaceutically acceptable salt of R-3 hydroxybutryrate.

87. The composition of claim 73, which is formulated for administration to a subject in a sustained release dosage or a controlled release dosage, and/or wherein the one or more ketone bodies are released from the microbead in a sustained and/or a controlled way upon administration to a subject.

88. The composition of claim 73, wherein the composition is formulated for use in the treatment of at least one neurological or neurodegenerative disease.

89. The composition of claim 88, wherein the at least one neurological disease is epilepsy and/or migraine, and/or the neurodegenerative disease is Alzheimer's disease.

90. A food product or food supplement comprising the composition of claim 73.

91. The food product or food supplement of claim 90, wherein said food product or food supplement is formulated for increasing cognitive performance, decreasing food craving, decreasing body weight, decreasing the fat to body weight ratio, maintaining or improving muscle power, and/or improving athletic performance and/or endurance of a subject.

92. A method for producing a composition of claim 73, wherein the method comprises the steps of
(a) preparing a denatured protein solution,
(b) combining a ketone body with the protein solution of step (a), wherein the ketone body is selected from the group consisting of
(i) beta-hydroxybutyric acid or beta-hydroxybutyrate (bHB),
(ii) acetoacetate (AcAc),
(iii) a precursor of bHB and/or AcAc,
(iv) a compound comprising an acetoacetyl- and/or 3-hydroxybutyrate moiety, and
(v) a pharmaceutically acceptable salt of any one of (a) to (d),
(c) extruding the mix of step (b) through an orifice, thereby forming microdroplets that free-fall in into a polymerization bath,
(d) curing the microdroplets formed in step (c) in the polymerization bath, thereby forming microbeads, and
(e) drying the microbeads of step (d), thereby producing said composition.
